(19) United States
(12) Patent Application Publication
(10) Pub. No.: US 2006/0206049 A1
(43) Pub. Date: Sep. 14, 2006

(54) MEMS flow module with piston-type pressure regulating structure

(76) Inventors: M. Steven Rodgers, Albuquerque, NM (US); Jeffry J. Sniegowski, Tijeras, NM (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/080,075

(22) Filed: Mar. 14, 2005

(51) Int. Cl. A61M 5/00

(52) U.S. Cl. 604/8

(57) ABSTRACT

Various embodiments of MEMS flow modules that regulate flow or pressure by the axial movement of a flow regulating or controlling structure are disclosed. One such MEMS flow module (40) has a regulator (66) that is aligned with and spaced from a first flow port (52) through a first plate (50). The regulator (66) is structurally interconnected with a flexible third plate (80). When the regulator (66) experiences at least a certain differential pressure, the regulator (66) moves at least generally axially away from the first plate (50) by a flexing of the third plate (80) at least generally away from the first plate (50). Increasing the spacing between the regulator (66) and the first plate (50) accommodates an increased flow or flow rate through the MEMS flow module (40).

### FIELD OF THE INVENTION

The present invention generally relates to the field of microfabricated devices and, more particularly, to a MEMS flow module that uses a piston-type structure to provide at least a pressure regulation function.

### BACKGROUND OF THE INVENTION

High internal pressure within the eye can damage the optic nerve and lead to blindness. There are two primary chambers in the eye—an anterior chamber and a posterior chamber that are generally separated by a lens. Aqueous humor exists within the anterior chamber, while vitreous humor exists in the posterior chamber. Generally, an increase in the internal pressure within the eye is caused by more fluid being generated within the eye than is being discharged by the eye. The general consensus is that it is the fluid within the anterior chamber of the eye that is the main contributor to an elevated intraocular pressure.

One proposed solution to addressing high internal pressure within the eye is to install an implant. Implants are typically directed through a wall of the patient's eye so as to fluidly connect the anterior chamber with an exterior location on the eye. There are a number of issues with implants of this type. One is the ability of the implant to respond to changes in the internal pressure within the eye in a manner that reduces the potential for damaging the optic nerve. Another is the ability of the implant to reduce the potential for bacteria and the like passing through the implant and into the interior of the patient's eye.

### BRIEF SUMMARY OF THE INVENTION

The present invention is generally embodied by what may be characterized as a MEMS flow module that provides at least a pressure regulation function. The use of the term “flow” in the description of the invention does not mean or require that a flow regulation function be provided in the form of providing a certain or desired a flow rate. Instead, the term “flow” is used in the description of the invention simply to identify that the invention accommodates a flow through the MEMS module, for instance to accommodate a different flow to provide a desired pressure regulation function.

A first aspect of the present invention is embodied by a MEMS flow module. This MEMS flow module includes a first film or plate having a first flow port and a second film or plate having a second flow port. A regulator is disposable in the second flow port such that the second plate and the flow port are disposed in a substantially common plane in the absence of at least a certain pressure differential across the MEMS flow module. The regulator is movable relative to both the first and second plates to change a magnitude of a spacing between the regulator and the first plate in response to at least a certain change in a differential pressure across the MEMS flow module.

Various refinements exist of the features noted in relation to first aspect of the present invention. Further features may also be incorporated in the first aspect of the present invention as well. These refinements and additional features may exist individually or in any combination. The regulator is movable in response to the development of at least a certain change in a differential pressure across the MEMS flow module as noted. Although this “certain change” in the differential pressure may be of any appropriate magnitude, preferably the regulator moves anytime the differential pressure is greater than zero, and furthermore preferably the regulator moves anytime there is any change in the differential pressure. All subsequent references herein to a “certain change” in the differential pressure or the like will be in accordance with the foregoing unless otherwise noted.

The regulator may move at least generally axially in response to at least a certain change in a differential pressure across the MEMS flow module, and more specifically across the regulator. It may be desirable to include a travel limiter or the like to provide a limit as to how far the regulator may move away from the first plate. Movement of the regulator away from the first plate in response to at least a certain pressure increase on the side of the regulator that faces the first plate, compared to the pressure on its opposite side, may accommodate an increase in the flow or flow rate through the MEMS flow module. Preferably the development of at least a certain change in the differential pressure across the regulator will provide greater than a linear increase in the flow rate through the MEMS flow module.

In one embodiment, movement of the regulator provides pressure regulation capabilities. In another embodiment, the MEMS flow module provides pressure regulation for a flow passing through the first flow port in a first direction, and acts at least similar to a check valve by at least generally restricting or impeding a flow through the MEMS flow module in a second direction that is opposite to the noted first direction. Consider the case where the MEMS flow module is used in an implant to relieve intraocular pressure in a patient's eye, and where the MEMS flow module is disposed in a flow path between the anterior chamber of the patient's eye and another drainage location or discharge region (e.g., exteriorly of the eye; another location within the eye or body). The first flow port may define an inlet to the MEMS flow module for a flow from the anterior chamber. The MEMS flow module may be used to regulate a flow of fluid out of the anterior chamber of the patient's eye in a manner that regulates the pressure in the anterior chamber in a desired manner, and may at least substantially restrict or impede a flow from the drainage location back through the MEMS flow module and into this anterior chamber. The MEMS flow module may be designed for a laminar flow therethrough in this and other instances, although the MEMS flow module may be applicable to a turbulent flow therethrough as well.

The first plate and the second plate may be disposed in a spaced relationship and interconnected by at least one first annular wall. “Annular” in relation to the first annular wall and other components described herein as being “annular” herein, means that the particular structure extends a full 360 degrees about a common reference point, and thereby does not limit the particular structure to having a circular configuration. This first annular wall may surround at least part of a flow path through the MEMS flow module so as to define at least one “radial” seal (e.g., to at least reduce the potential for fluid escaping from the MEMS flow module through the space between the first and second plates). Using multiple, radially spaced first annular walls would thereby provide redundant radial seals. The first and second flow ports would then be located inwardly of each such first annular wall in a radial or lateral dimension. One or more additional structural interconnections of any appropriate size, shape, configuration, and arrangement may exist between the first and second plates to provide a desired degree of rigidity for the MEMS flow module. The first plate could also be formed directly on or disposed in interfacing relation with the second plate to increase the rigidity of the MEMS flow module as well, with the first and second flow ports being fluidly interconnected in any appropriate manner.

The regulator may be of any appropriate size, shape, and/or configuration. The regulator should be operative to move so as to control pressure by accommodating a flow or a change in flow through the MEMS flow module. Such movement of the regulator may be at least generally orthogonal to the plane defined by the second plate (e.g., at least generally axial motion). In this regard, the spacing between the regulator and the first plate may be substantially constant about the perimeter of the regulator (e.g., the regulator and first plate may be parallel to each other). The orientation of the regulator may also be at least substantially maintained during its movement to provide a pressure regulation function.

The regulator may be sized such that it at least generally overlays the first flow port or an area/region through which a flow is discharged from the first flow port, at least when the regulator is disposed in the substantially common plane with the second plate. This may permit the regulator to substantially restrict or impede a flow across the MEMS flow module in the absence of at least a certain pressure differential across the MEMS flow module. In one embodiment, an outside perimeter of the regulator and a sidewall of the second plate that defines the second flow port are separated by an annular gap when the regulator is disposed within the second flow port. The width of this gap may be constant or otherwise. The sidewall of the second plate that defines this gap may be of any desired configuration and disposed in any desired orientation as well (e.g., it may be disposed perpendicularly to the primary surfaces of the second plate (e.g., in the form of a cylindrical surface); it may be disposed at an inclined angle relative to the primary surfaces of the second plate so as to be “tapered” in the direction of the flow therethrough (e.g., in the form of a frustumly-shaped surface)). One or more sidewall configurations may provide one or more desired flow characteristics. For instance, the sidewall of the second plate that defines the second flow port may be shaped to provide a reduced flow resistance, to thereby accommodate an increased flow through the second flow port. It also may be possible for the regulator to contact the sidewall of the second plate that defines the second flow port at one or more locations, although having such contact is less preferred.

The regulator may be fabricated in any manner that allows for it to be disposed within the second flow port such that, in the absence of at least a certain pressure differential across the MEMS flow module, the regulator and second plate are disposed in a substantially common plane. For instance, the second plate and the regulator may exist in a common fabrication level. Furthermore, the regulator and the second plate may be free of any structural interconnections in this common fabrication level. In this regard, the regulator may be movably supported relative to the first and second plates by one or more additional structures.

In one embodiment, the regulator is movably supported by a third film or plate that is incorporated in the MEMS flow module such that the second plate is disposed somewhere between the first and third plates. That portion of the third plate that allows the regulator to move should be spaced from the second plate. One or more third flow ports may extend through the third plate to accommodate a flow through the MEMS flow module.

The second plate and the third plate may be disposed in a spaced relationship and interconnected by at least one second annular wall. This second annular wall may surround at least part of a flow path through the MEMS flow module so as to define at least one “radial” seal (e.g., to at least reduce the potential for fluid escaping from the MEMS flow module through the space between the second and third plates in the radial or lateral dimension). Using multiple, radially spaced second annular walls would thereby provide redundant radial seals. The second and third flow ports would then be located inwardly of each such second annular wall in a radial or lateral dimension. One or more additional structural interconnections of any appropriate size, shape, configuration, and arrangement may exist between the second and third plates to provide a desired degree of rigidity for the MEMS flow module. The second plate could also be formed directly on or disposed in interfacing relation with a “stationary portion” of the third plate (e.g., any portion of the third plate that does not move to any significant degree to accommodate movement of the regulator) in order to increase the rigidity of the MEMS flow module as well.

To effect movement of the regulator, the third plate may be structurally interconnected with the regulator in any appropriate manner. In one embodiment, the third plate is in the form of a diaphragm that is “unsupported” inwardly of the above-noted second annular wall. In this case, an anchor or other appropriate mechanical link may extend from the regulator down to such an unsupported portion of the third plate. In this case, the development of at least a certain pressure differential across the MEMS flow module (more specifically across the regulator) may flex the unsupported portion of the third plate away from the second plate to allow the regulator to move (e.g., at least generally axially) relative to the first and second plates. Accordingly, a spacing may be created between the regulator and the first flow port (or the region through which a flow from the first flow port is discharged prior to encountering the regulator), or the size of this spacing may be increased, all to permit an increased flow through the MEMS flow module.

As the magnitude of the noted pressure differential is reduced, the third plate may move back at least towards its initial/static position (e.g., wherein the regulator is substantially coplanar with the second plate) using the elastic or spring forces that were created and stored within the third plate by flexing away from the second plate. That is, the internal stresses caused by flexing the third plate of the MEMS flow module away from the second plate may provide a restoring force that at least contributes to moving the regulator back toward or all the way back to its static or home position.

In another embodiment, the third plate is in the form of an annular support and one or more flexible or elongated support members are utilized to moveably support the regulator relative to the first and second plates. Each such support member may be of any appropriate size, shape, and/or configuration. A space may exist at least along each side of each support member to define a third flow port for accommodating a flow through the MEMS flow module (e.g., the entire region between adjacent pairs of support members may be an open space that defines a third flow port; a discrete channel may exist along each side of each support member). The annular support may be spaced from the second plate and interconnected therewith by at least one second annular wall of the above-noted type. The second plate could also be fabricated directly on or disposed in interfacing relation with this annular support.

Each support member movably interconnects the regulator with the annular support of the third plate. For instance, a first end of each support member may be appropriately interconnected with the annular support (e.g., defining a fixed end of the support member), and a second end of each support member (e.g., a free end of the support member) may be appropriately interconnected with the regulator. Although it may be possible for the free end of each support member to be directly attached to the regulator, more typically an appropriate linking structure will extend between the regulator and the free ends of the various support members. For instance, the various support members may converge at a location to which this linking structure extends. In one embodiment, the support members are equally spaced from each other and are each disposed along a radii emanating from a common center.

The various support members will flex when the MEMS flow module is exposed to at least a certain differential pressure to allow the regulator to move relative to the first and second plates. Preferably the support members elastically deform. In this case, the attempt of each support member to return toward its undeformed state may provide a restoring force that at least contributes to the movement of the regulator back toward its “home” or “differential pressure set-point” position (e.g., the position of the regulator when there is no differential pressure across the regulator) as the magnitude of the differential pressure is reduced. The noted differential pressure “set-point” may be any appropriate value, including zero. Preferably, the regulator moves in response to any differential pressure greater than zero or when there is any change in the differential pressure for that matter.

The annular support and each support member may be fabricated in a common fabrication level. This fabrication level may be a separate one from the common fabrication level in which the second plate and regulator may be fabricated. In one embodiment, the annular support and the various support members are disposed in a substantially common plane in the absence of at least a certain differential pressure across the MEMS flow module.

In one embodiment, the position of the regulator is based upon the differential pressure to which it is exposed, and the position of the regulator will at least partially determine the flow rate through the MEMS flow module. Generally, the flow rate through the MEMS flow module will increase as the spacing between the regulator and the first plate increases, and will decrease as the spacing between these same components decreases. Preferably, the flow rate through the MEMS flow module will increase greater than proportionally for a corresponding increase in the pressure differential across the MEMS flow module.

The above-noted movement of the regulator in response to a pressure differential across the MEMS flow module is itself subject to a number of characterizations. One is that the regulator may be operative to move in at least two different directions. For instance, the regulator may move at least generally away from the first plate, which may allow for increasing the volume of a flow channel associated with and downstream of the first flow port. The regulator may also move at least generally toward the first plate, which may allow for reducing the volume of this same flow channel and/or substantially restricting or impeding a flow through the first flow port.

Another characterization is that a flow path having a volume greater than zero may always be present through the MEMS flow module. For instance, the regulator may be spaced relative to the first plate such that a flow path segment having at least a predetermined minimum size may be constantly maintained for receiving a flow from the first flow port in a first direction or directing a flow into and through the first flow port in a second direction that is opposite of the first direction. An appropriate mechanical “stop” could be used to provide/maintain a minimum spacing between the regulator and the first plate. Such a flow path segment may remain open even in the absence of a differential pressure that is adequate to flex a supporting structure associated with the regulator, again where a flexing of the supporting structure allows the regulator to move away from the first plate. However, another option would be for the regulator to actually preclude any flow through the first flow port until the development of at least a certain differential pressure.

The first flow port may be of any appropriate size and/or shape. Further, the first plate may include a plurality of first flow ports that pass through the first plate. Likewise, the MEMS flow module may include a second plate having a plurality of second flow ports that may correspond with the plurality of first flow ports. Further, the MEMS flow module may include a plurality of regulators disposed within the plurality of second flow ports, wherein each first flow port has a corresponding second flow port and a corresponding regulator. Accordingly, such a plurality of regulators may utilize any support structure that permits each regulator to move relative to their corresponding first flow port.

Each first flow port may further include an associated flow-restricting structure. This flow-restricting structure may extend from the first plate and proceed toward the regulator, and terminate prior to reaching the regulator. The flow-restricting structure may reduce the size of a space through which a flow must progress after passing through the first flow port, and the size of which is determined at least in part by the position of the regulator. In one embodiment, the flow-restricting structure terminates prior to reaching the regulator. The flow-restricting structure may be of any appropriate form, such as an annular wall or a plurality of flow-restricting segments that are appropriately spaced from each other. Alternatively, such a flow-restricting structure may extend from the regulator toward the first plate. Another option would be for the regulator to include a plug that is at least aligned with the first flow port. Such a plug could simply be disposed “over” the first flow port, or such a plug could actually extend into the first flow port (preferably remaining spaced therefrom).

A second aspect of the present invention is embodied by a MEMS flow module. The MEMS flow module includes a first fabrication level having a first film or plate that includes a first flow port, as well as a second fabrication level that includes both a second film or plate and a regulator. That is, there are at least two separate fabrication levels. A second flow port is associated with the second plate, and the regulator fluidly communicates with the first flow port. The regulator is moveable relative to the first and second plates to change a magnitude of a spacing of the regulator from the first plate in response to at least a certain change in a differential pressure across the MEMS flow module. The various features discussed above in relation to the first aspect may be used by this second aspect, individually or in any combination. As noted above, although this “certain” differential pressure may be of any appropriate magnitude, preferably the regulator moves anytime the differential pressure is greater than zero, and furthermore preferably the regulator will move anytime there is any change in the differential pressure.

In addition to the foregoing, the second plate may be in the form of an annular support, and a plurality of support members that extend from this annular support to the regulator. These support members allow the regulator to move relative to both the first plate and the annular support of the second plate (e.g., by a deflection or deformation). Preferably these support members elastically deflect or deform so as to move the regulator at least back toward its static or home position upon a reduction of the differential pressure. In any case, the plurality of support members may exist in the second fabrication level as well, and further may be of any appropriate size, shape, and configuration. Any number of support members may be utilized as well. The space between an adjacent pair of support members may define the second flow port. A plurality of second flow ports of this type may be provided as well (e.g., by using three or more of the noted support members).

A third aspect of the present invention is embodied by a MEMS flow module. The flow module includes a first film or plate having a first flow port, a second film or plate having a second flow port, and a regulator that fluidly communicates with the first flow port. The regulator is sized such that an outside perimeter of the regulator and a sidewall of the second plate that defines the second flow port are separated by an annular gap when the regulator is disposed within the second flow port. Further, the regulator is moveable relative to the first and second plates to change the magnitude of a spacing of the regulator from the first plate in response to at least a certain change in a differential pressure across the MEMS flow module. The various features discussed above in relation to the first aspect may be used by this third aspect, individually or in any combination. As noted above, although this “certain” differential pressure may be of any appropriate magnitude, preferably the regulator moves anytime the differential pressure is greater than zero, and furthermore preferably the regulator will move anytime there is any change in the differential pressure.

A fourth aspect is embodied by a MEMS flow module. The MEMS flow module includes first, second, and third films or plates, each having at least at least one flow port extending therethrough. The MEMS flow module further includes at least one regulator that is located somewhere between the first and third plates (e.g., disposable within a second flow port through the second plate). At least part of the third plate compliantly supports the regulator(s) to allow the regulator(s) to move at least generally axially. Movement of the regulator(s) away from the first plate, in response to an increase in the pressure acting on the side of the regulator(s) that communicates with a corresponding first flow port through the first plate, versus the pressure acting on the side of the regulator(s) that communicates with a corresponding third flow port through the third plate, accommodates an increased flow through the MEMS flow module in a direction that proceeds through the first flow port, through the second flow port, and then through the third flow port. The various features discussed above in relation to the first aspect may be used by this fourth aspect, individually or in any combination.

A fifth aspect of the present invention is embodied by a MEMS flow module. A film or plate includes at least one flow port. A regulator is disposable within this flow port. The regulator is movable relative to the plate, including within the flow port, in response to experiencing at least a certain differential pressure across the MEMS flow module.

Various refinements exist of the features noted in relation to fifth aspect of the present invention. Further features may also be incorporated in the fifth aspect of the present invention as well. These refinements and additional features may exist individually or in any combination. The regulator is movable in response to experiencing at least a certain differential pressure across the MEMS flow module as noted. Although this “certain differential pressure” may be of any appropriate magnitude, preferably the regulator moves anytime the differential pressure is greater than zero, and furthermore preferably the regulator moves anytime there is any change in the differential pressure.

The regulator preferably moves at least generally along an axial path in response to experiencing at least a certain differential pressure. Any way of supporting the regulator so as to move in this manner may be utilized (e.g., by compliantly supporting the regulator relative to the plate). In one embodiment, the regulator and the plate are disposed at least substantially within a common plane in the absence of any differential pressure across the MEMS flow module. For instance, the plate and the regulator may exist in a common fabrication level.

Preferably an annular space or gap exists between the perimeter of the regulator and a sidewall of the plate that defines the flow port, at a time when the regulator is disposed within the flow port. This annular space or gap may be of an at least substantially constant width about the entire perimeter of the regulator. This annular gap may also be of any appropriate configuration (e.g., the sidewall of the plate that defines this flow port may be a cylindrical surface; the sidewall of the plate that defines this flow port may be frustumly-shaped). One or more sidewall configurations may provide one or more desired flow characteristics. For instance, the sidewall of the plate that defines the flow port may be shaped to provide a reduced flow resistance, to thereby accommodate an increased flow through this flow port. In any case, as the regulator becomes more offset relative to the plate, a flow resistance decreases. This then accommodates an increased flow or flow rate through the MEMS flow module.

Surface micromachining is the preferred technology for fabricating the MEMS flow modules described herein. In this regard, the various plates and regulators of the MEMS flow modules described herein each may be fabricated from one or more layers or films, where each layer or film has a thickness of no more than about 10 microns in one embodiment, and more typically a thickness within a range of about 1 micron to about 3 microns in another embodiment. Each of the MEMS flow modules described herein may be fabricated in at least two different or separate fabrication levels (hereafter a first fabrication level and a second fabrication level). “Fabrication level” corresponds with what may be formed by a deposition of a structural material before having to form any overlying layer of a sacrificial material (e.g., from a single deposition of a structural layer or film). The second plate and/or the regulator discussed herein may be fabricated at least in the first fabrication level, while the first plate discussed herein may be fabricated in at least the second fabrication level. It should be appreciated that the characterization of the second plate and/or regulator being in the “first fabrication level” and the first plate being in the “second fabrication level” by no means requires that the first fabrication level be that which is deposited “first”, and that the second fabrication level be that which is deposited “second.” Moreover, it does not require that the first fabrication level and the second fabrication level be immediately adjacent to each other. These MEMS flow modules may be fabricated on an appropriate substrate and where the first plate is fabricated in one structural layer that is disposed somewhere between the substrate and another structural layer in which the second plate and/or regulator is fabricated, or vice versa.

The regulator/second plate and the first plate each may exist in a single fabrication level or may exist in multiple fabrication levels. In the above-noted first instance, a deposition of a structural material in a single fabrication level may define an at least generally planar layer. Another option regarding the first instance would be for the deposition of a structural material in a single fabrication level to define an at least generally planar portion, plus one or more structures that extend down toward, but not to, the underlying structural layer at the underlying fabrication level. In either situation and prior to the release, in at least some cases there will be at least some thickness of sacrificial material disposed between the entirety of the regulator/second plate and the first plate.

Two or more structural layers or films from adjacent fabrication levels could also be disposed in direct interfacing relation as previously noted (e.g., one directly on the other). Over the region that is to define the first plate or second plate, this would require removal of at least some of the sacrificial material that is deposited on the structural material at one fabrication level before depositing the structural material at the next fabrication level (e.g. sacrificial material may be encased by a structural material, so as to not be removed by the release). Another option would be to maintain the separation between structural layers or films in different fabrication levels for the first plate and second plate, but provide an appropriate structural interconnection therebetween (e.g., a plurality of columns, posts, or the like extending between adjacent structural layers or films in different fabrication levels).

The MEMS flow modules described herein are preferably passive devices (no external electrical signal of any type required) and may be used for any appropriate application. Another characterization of these MEMS flow modules is that they are autonomous in that they are self-contained structures and require no external power. For instance, any of these MEMS flow modules may be disposed in a flow path of any type (e.g., between a pair of sources of any appropriate type, such as a man-made reservoir, a biological reservoir, and/or the environment), and further may be used for any appropriate application. That is, one or more of any of these MEMS flow modules could be disposed in a conduit that fluidly interconnects multiple sources (e.g., two or more), and each source may be either a man-made reservoir, a biological reservoir, the environment, or any other appropriate source. One example would be to dispose one or more of these MEMS flow modules in a conduit extending between the anterior chamber of an eye and a location that is exterior of the cornea of the eye. Another example would be to dispose one or more of these MEMS flow modules in a conduit extending between the anterior chamber of an eye and another location that is exterior of the sclera of the eye. Yet another example would be to dispose one or more of these MEMS flow modules in a conduit extending between the anterior chamber of an eye and another location within the eye (e.g., into Schlemm's canal) or body. In any case, any of these MEMS flow modules could be disposed directly into such a conduit, or one or more housings could be used to integrate any of these MEMS flow modules with the conduit. In each of these examples, the conduit would provide an exit path for aqueous humor when installed for a glaucoma patient. That is, each of these examples may be viewed as a way of treating glaucoma or providing at least some degree of control of the intraocular pressure.

Each of the MEMS flow modules described herein may be used in combination with a conduit to define an implant that is installable in a biological mass. This implant may be used to address pressure with a first body region. In this regard, the conduit may include a flow path that is adapted to fluidly interconnect with the first body region, and at least one MEMS flow module may be disposed within this flow path. In one embodiment, at least one housing is used to establish an interconnection or interface between the conduit and the MEMS flow module. For instance, the housing may be at least partially disposed within the conduit, and the MEMS flow module may interface with the housing. Although any appropriate implant application is contemplated, in one embodiment the implant is installable in a human eye to fluidly interconnect with an anterior chamber of the human eye for purposes of regulating intraocular pressure.

### BRIEF DESCRIPTION OF THE SEVERAL VIEWS OF THE DRAWING

FIG. 1 is a side view of a plurality of layers that may be used by one embodiment of a surface micromachining fabrication technique.

FIG. 2A is a perspective view of a first embodiment of a MEMS flow module.

FIG. 2B is a cross-sectional, exploded, perspective view of first and second plates, as well as a regulator, of the MEMS flow module of FIG. 2A.

FIG. 2C is a cross-sectional, exploded, perspective view of the second plate, the regulator, and a third plate of the MEMS flow module of FIG. 2A.

FIG. 2D is a cross-sectional view through the first plate, second plate, and regulator of the MEMS flow module of FIG. 2A.

FIG. 2E is a perspective bottom view of a second embodiment of a compliant support for the regulator of the MEMS flow module of FIG. 2A.

FIG. 2F is a perspective bottom view of a third embodiment of a compliant support for the regulator of the MEMS flow module of FIG. 2A.

FIG. 3A is a cross-sectional view of a second embodiment of a MEMS flow module and in a position when there is no differential pressure across the MEMS flow module.

FIG. 3B is a representative position of the MEMS flow module of FIG. 3A when exposed to a differential pressure.

FIG. 3C is an enlarged view of the flow port used by the MEMS flow module of FIG. 3A, and that may be used by the other MEMS flow modules described herein.

FIG. 3D is an enlarged view of one variation of the flow port used by the MEMS flow module of FIG. 3A, and that may be used by the other MEMS flow modules described herein.

FIG. 3E is an enlarged view of another variation of the flow port used by the MEMS flow module of FIG. 3A, and that may be used by the other MEMS flow modules described herein.

FIG. 3F is an enlarged view of another variation of the flow port used by the MEMS flow module of FIG. 3A, and that may be used by the other MEMS flow modules described herein.

FIG. 3G is an enlarged view of another variation of the flow port used by the MEMS flow module of FIG. 3A, and that may be used by the other MEMS flow modules described herein.

FIG. 3H is an enlarged view of another variation of the flow port used by the MEMS flow module of FIG. 3A, and that may be used by the other MEMS flow modules described herein.

FIG. 4A is a cross-sectional view of a third embodiment of a MEMS flow module.

FIG. 4B is a top view of the MEMS flow module of FIG. 4A.

FIG. 5 is a perspective view of a fourth embodiment of a MEMS flow module that uses multiple regulators.

FIG. 6 is a cross-sectional view of a one embodiment of a flow restrictor for an etch release hole that may be utilized by any of the MEMS flow modules described herein.

FIG. 7 is an exploded, perspective view of one embodiment of a flow assembly that uses a MEMS flow module.

FIG. 8 is a perspective view of the flow assembly of FIG. 7 in an assembled condition.

FIG. 9A is an exploded, perspective of another embodiment of a flow assembly that uses a MEMS flow module.

FIG. 9B is a perspective view of the flow assembly of FIG. 9A in an assembled condition.

FIG. 10A is an exploded, perspective of another embodiment of a flow assembly that uses a MEMS flow module.

FIG. 10B is a perspective view of the flow assembly of FIG. 10A in an assembled condition.

FIG. 11A is a schematic of one embodiment of a glaucoma or intraocular implant that may use any of the MEMS flow modules described herein.

FIG. 11B is a cross-sectional view of one embodiment of a glaucoma or intraocular implant or shunt that is used to relieve pressure within the anterior chamber of the eye, and that may utilize any of the MEMS flow modules described herein.

### DETAILED DESCRIPTION OF THE INVENTION

The present invention will now be described in relation to the accompanying drawings that at least assist in illustrating its various pertinent features. Generally, the devices described herein are microfabricated. There are a number of microfabrication technologies that are commonly characterized as “micromachining,” including without limitation LIGA (Lithographie, Galvonoformung, Abformung), SLIGA (sacrificial LIGA), bulk micromachining, surface micromachining, micro electrodischarge machining (EDM), laser micromachining, 3-D stereolithography, and other techniques. Hereafter, the term “MEMS device”, “microfabricated device,” or the like means any such device that is fabricated using a technology that allows realization of a feature size of 10 microns or less.

Surface micromachining is currently the preferred fabrication technique for the various devices to be described herein. One particularly desirable surface micromachining technique is described in U.S. Pat. No. 6,082,208, that issued Jul. 4, 2000, that is entitled “Method For Fabricating Five-Level Microelectromechanical Structures and Microelectromechanical Transmission Formed,” and the entire disclosure of which is incorporated by reference in its entirety herein. Surface micromachining generally entails depositing alternate layers of structural material and sacrificial material using an appropriate substrate (e.g., a silicon wafer) which functions as the foundation for the resulting microstructure. Various patterning operations (collectively including masking, etching, and mask removal operations) may be executed on one or more of these layers before the next layer is deposited so as to define the desired microstructure. After the microstructure has been defined in this general manner, all or a portion of the various sacrificial layers are removed by exposing the microstructure and the various sacrificial layers to one or more etchants. This is commonly called “releasing” the microstructure.

The term “sacrificial layer” as used herein means any layer or portion thereof of any surface micromachined microstructure that is used to fabricate the microstructure, but which does not generally exist in the final configuration (e.g. sacrificial material may be encased by a structural material at one or more locations for one or more purposes, and as a result this encased sacrificial material is not removed by the release). Exemplary materials for the sacrificial layers described herein include undoped silicon dioxide or silicon oxide, and doped silicon dioxide or silicon oxide (“doped” indicating that additional elemental materials are added to the film during or after deposition). The term “structural layer” as used herein means any other layer or portion thereof of a surface micromachined microstructure other than a sacrificial layer and a substrate on which the microstructure is being fabricated. Exemplary materials for the structural layers described herein include doped or undoped polysilicon and doped or undoped silicon. Exemplary materials for the substrates described herein include silicon. The various layers described herein may be formed/deposited by techniques such as chemical vapor deposition (CVD) and including low-pressure CVD (LPCVD), atmospheric-pressure CVD (APCVD), and plasma-enhanced CVD (PECVD), thermal oxidation processes, and physical vapor deposition (PVD) and including evaporative PVD and sputtering PVD, as examples.

In more general terms, surface micromachining can be done with any suitable system of a substrate, sacrificial film(s) or layer(s) and structural film(s) or layer(s). Many substrate materials may be used in surface micromachining operations, although the tendency is to use silicon wafers because of their ubiquitous presence and availability. The substrate is essentially a foundation on which the microstructures are fabricated. This foundation material must be stable to the processes that are being used to define the microstructure(s) and cannot adversely affect the processing of the sacrificial/structural films that are being used to define the microstructure(s). With regard to the sacrificial and structural films, the primary differentiating factor is a selectivity difference between the sacrificial and structural films to the desired/required release etchant(s). This selectivity ratio may be on the order of about 10:1, and is more preferably several hundred to one or much greater, with an infinite selectivity ratio being most preferred. Examples of such a sacrificial film/structural film system include: various silicon oxides/various forms of silicon; poly germanium/poly germanium-silicon; various polymeric films/various metal films (e.g., photoresist/aluminum); various metals/various metals (e.g., aluminum/nickel); polysilicon/silicon carbide; silicone dioxide/polysilicon (i.e., using a different release etchant like potassium hydroxide, for example). Examples of release etchants for silicon dioxide and silicon oxide sacrificial materials are typically hydrofluoric (HF) acid based (e.g., concentrated HF acid, which is actually 49 wt % HF acid and 51 wt % water; concentrated HF acid with water; buffered HF acid (HF acid and ammonium fluoride)).

The microfabrication technology described in the above-noted '208 patent uses a plurality of alternating structural layers (e.g., polysilicon and therefore referred to as “P” layers herein) and sacrificial layers (e.g., silicon dioxide, and therefore referred to as “S” layers herein). The nomenclature that is commonly used to describe the various layers in the microfabrication technology described in the above-noted '208 patent will also be used herein.

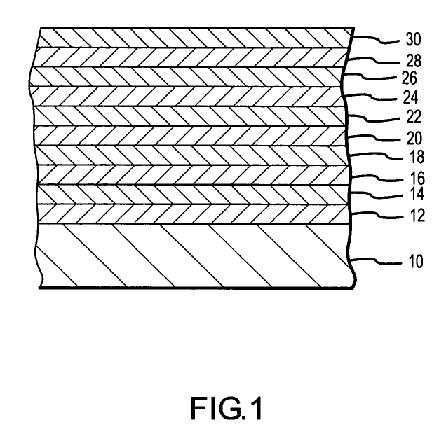

FIG. 1 generally illustrates one embodiment of layers on a substrate 10 that is appropriate for surface micromachining and in accordance with the nomenclature commonly associated with the '208 patent. Each of these layers will typically have a thickness of no more than about 10 microns, and more typically a thickness within a range of about 1 micron to about 3 microns. Progressing away from the substrate 10, the various layers are: a dielectric layer 12 (there may be an intermediate oxide layer between the dielectric layer 12 and the substrate 10 as well, which is not shown); a P₀ layer 14 (a first fabrication level); an S₁ layer 16; a P₁ layer 18 (a second fabrication level); an S₂ layer 20; a P₂ layer 22 (a third fabrication level); an S₃ layer 24; a P₃ layer 26 (a fourth fabrication level); an S₄ layer 28; and a P₄ layer 30 (a fifth fabrication level). In some cases, the S₂ layer 20 may be removed before the release such that the P₂ layer 22 is deposited directly on the P₁ layer 18, and such may hereafter be referred to as a P₁/P₂ layer. It should also be appreciated that one or more other layers may be deposited on the P₄ layer 30 after the formation thereof and prior to the release, where the entirety of the S₁ layer 16, S₂ layer 20, S₃ layer 24, and S₄ layer 28 may be removed (although portions of one or more of these layers may be retained for one or more purposes if properly encased so as to be protected from the release etchant). It should also be appreciated that adjacent structural layers may be structurally interconnected by forming cuts or apertures through the entire thickness of a particular sacrificial layer before depositing the next structural layer. In this case, the structural material will not only be deposited on the upper surface of the particular sacrificial layer, but will be deposited in these cuts or apertures as well (and will thereby interconnect a pair of adjacent, spaced, structural layers).

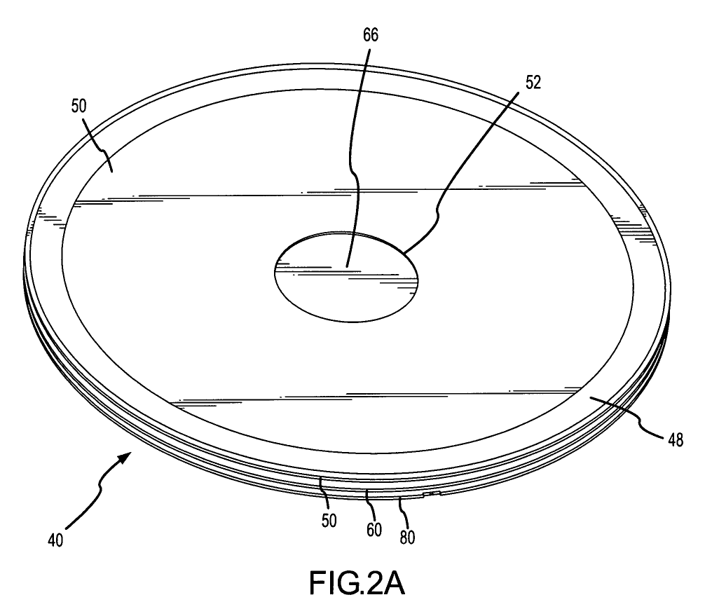

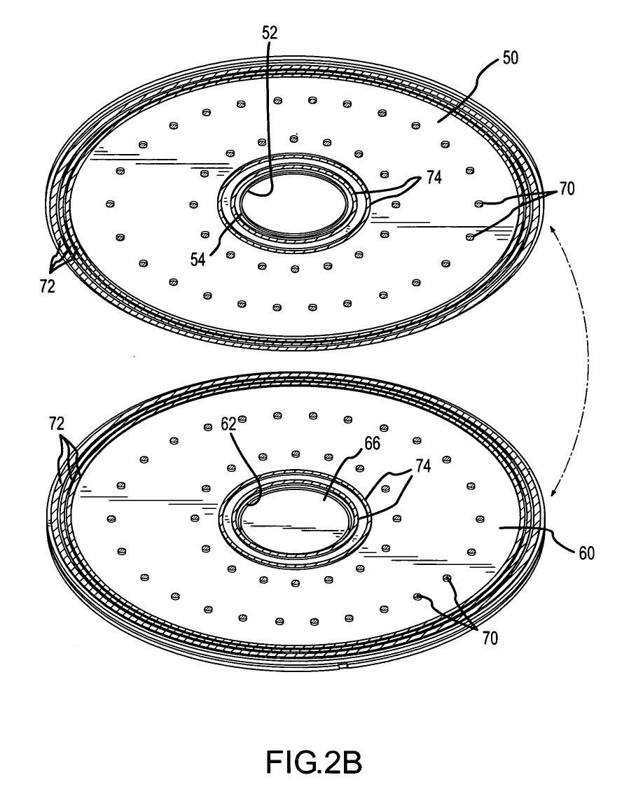

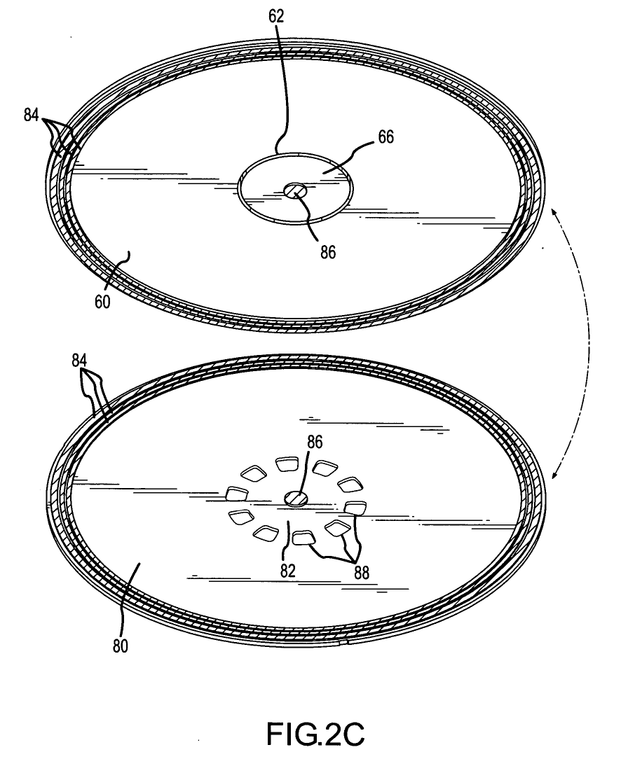

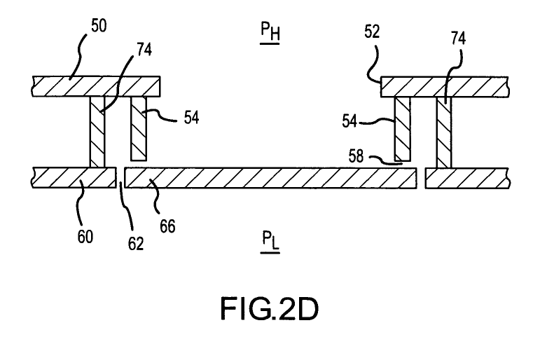

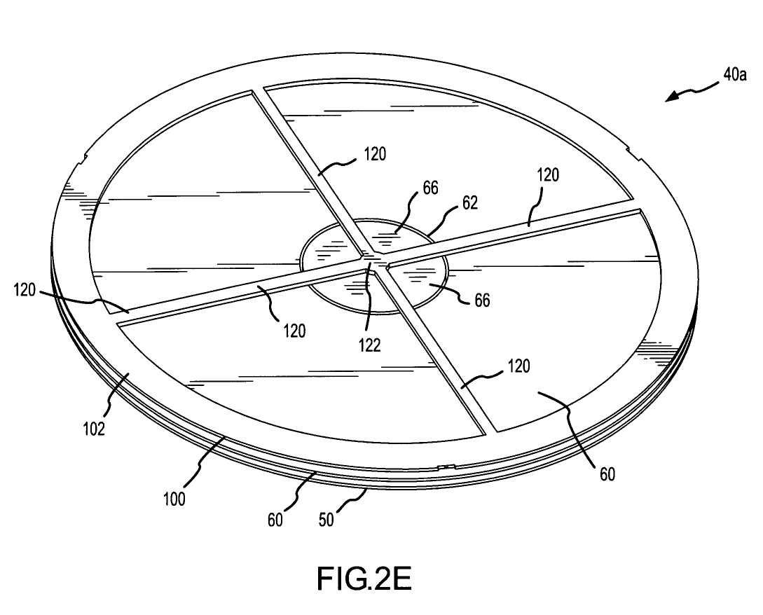

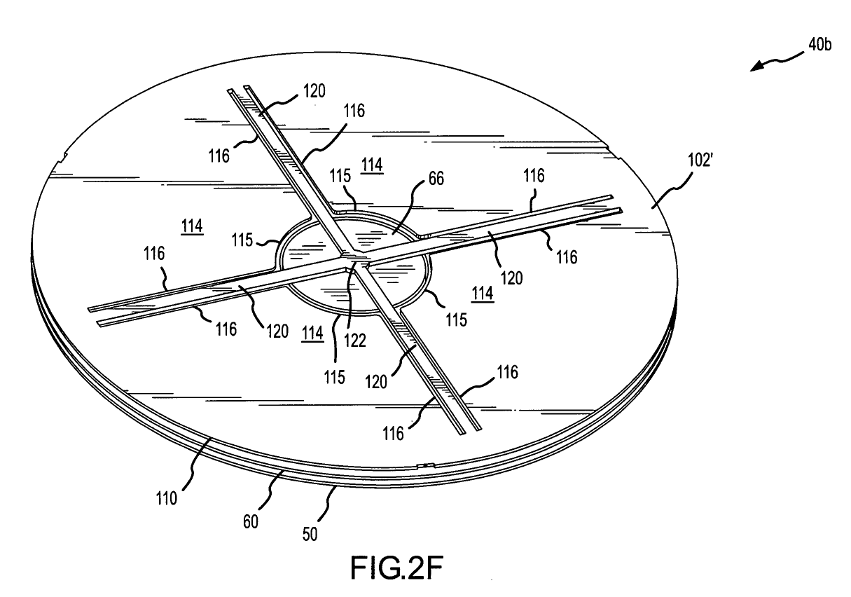

The general construction of one embodiment of a MEMS flow module (a MEMS device) is illustrated in FIGS. 2A-D, is identified by reference numeral 40, and provides pressure regulation capabilities, filtration capabilities, or both. Typically, the MEMS flow module 40 will be used for a pressure regulation application. Although the MEMS flow module 40 is illustrated as having a circular configuration in plan view, any appropriate configuration may be utilized and in any appropriate size.

As shown in FIGS. 2A-2C, the MEMS flow module 40 includes a first plate 50 (e.g., fabricated in P₄ layer 30) having a first flow port 52 that extends completely through the first plate 50, a second plate 60 (e.g., fabricated in P₃ layer 26) having a second flow port 62 that extends through the second plate 60, and a third plate 80 that compliantly supports a piston-type regulator 66 (typically within the second flow port 62) and that includes a plurality of third flow ports 88. More specifically, the third plate 80 supports the regulator 66 in a certain spaced relationship relative to the first plate 50 (e.g., in at least a substantially co-planar relationship with the second plate 60) until the development of at least a certain differential pressure across the MEMS flow module 40 (more specifically across the regulator 66). It would be typical to configure the MEMS flow module 40 (as well as the other MEMS flow modules to the described herein) to allow a target flow rate for a target differential pressure. The flow rate through the MEMS flow module 40 at other differential pressures would depend on the various characteristics of the MEMS flow module 40.

The third plate 80 is operative to flex in response to the development of at least a certain pressure differential across the MEMS flow module 40 such that the regulator 66 is able to move at least generally axially away from the first plate 50 and first flow port 52. Although the amount of differential pressure required to flex the third plate 80 may be of any appropriate magnitude, preferably the third plate 80 will flex to at least some degree anytime the differential pressure across the MEMS flow module 40 is greater than zero or anytime there is any change in the differential pressure. As such, the regulator 66 will then preferably move anytime the differential pressure across the MEMS flow module 40 is greater than zero or anytime there is any change in the differential pressure.

Movement of the regulator 66 away from the first plate 50 accommodates an increase in a fluid flow or flow rate through the MEMS flow module 40. That is, increasing the spacing between the regulator 66 and the first plate 50 (in response to an increasing differential pressure) increases the flow rate through the MEMS flow module 40, while decreasing the spacing between the regulator 66 and the first plate 50 (in response to a decreasing differential pressure) decreases the flow rate through the MEMS flow module 40. It may be desirable to incorporate one or more structures to maintain a minimum spacing between the first plate 50 and the regulator 66, to incorporate one or more structures to provide a maximum spacing between the first plate 50 and the regulator 66, or both (not shown).

The first plate 50 will typically be oriented as the “inlet side” or “high pressure side” of the MEMS flow module 40. Any appropriate size, shape, and/or configuration may be utilized for the first plate 50. As noted, the first flow port 52 extends completely through the first plate 50. In the illustrated embodiment, the first flow port 52 is centrally disposed relative to the first plate 50, and its center is aligned with the center of the regulator 66. It may be such that the first flow port 52 could be disposed at other locations and in other positions relative to the regulator 66. Generally, the first flow port 52 should be positioned relative to the regulator 66 so that the first flow port 52 exposes at least part of the regulator 66 to a pressure acting on the first plate 50. What is at least generally required is for the regulator 66 to fluidly communicate with the first flow port 52.

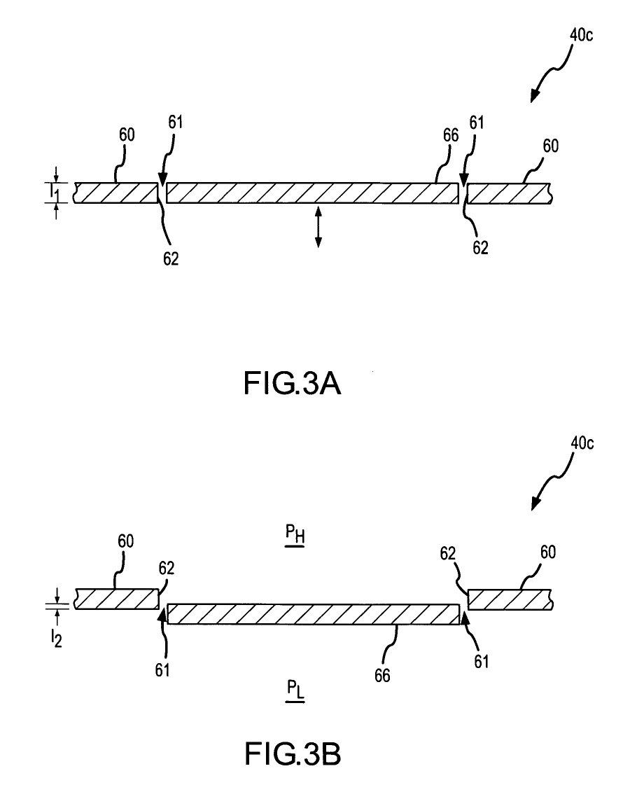

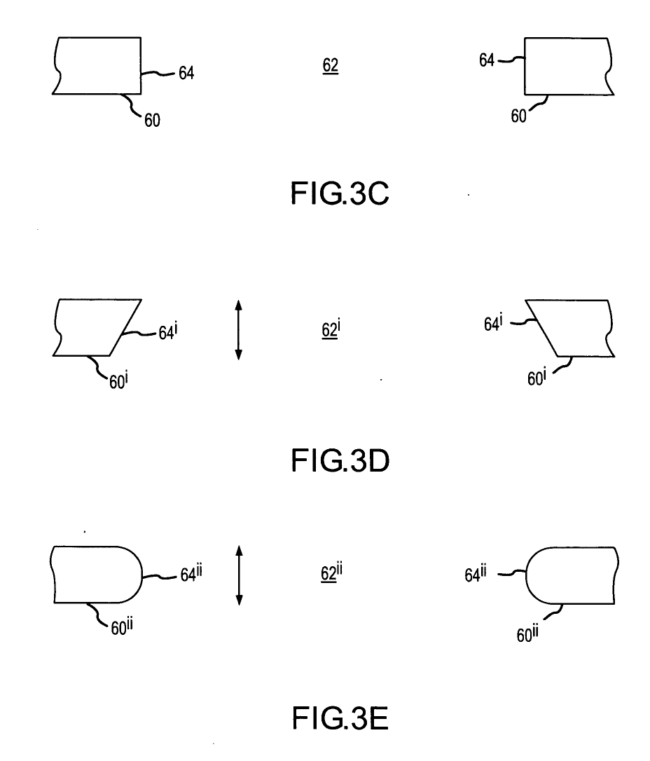

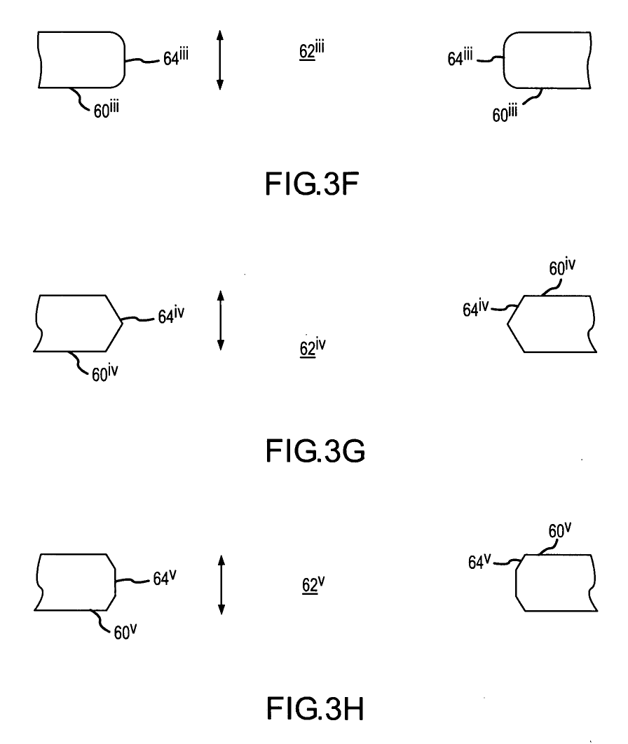

As best shown in FIGS. 2B-2D, the regulator 66 may be fabricated in a common fabrication level with the second plate 60 (e.g., both being fabricated in the P₃ layer 26). The regulator 66 is sized for receipt within the second flow port 62 of the second plate 60 such that the second plate 60 and the regulator 66 may be disposed in at least substantially coplanar relation until the development of at least a certain differential pressure across the MEMS flow module 40. Preferably, an annular space will exist between the perimeter of the regulator 66 and a sidewall of the second plate 60 that defines the second flow port 62 when the regulator 66 is at least partially disposed within the second flow port 62. It could be such that the width of this space is not constant (about the perimeter, along its length, or both) or that the regulator 66 could actually contact the sidewall of the second plate 60 that defines the second flow port 62 at one or more locations. The sidewall of the second plate 60 that defines the second flow port 62 also may be of any appropriate configuration (e.g., cylindrical, frustumly-shaped or conically-shaped). One or more sidewall configurations may provide one or more desired flow characteristics. For instance, the sidewall of the second plate 60 that defines the second flow port 62 may be shaped to provide a reduced flow resistance, to thereby accommodate an increased flow through the second flow port 62 (e.g., see the following discussion of FIGS. 3C-H). In any case, this spacing between the regulator 66 and the sidewall of the second plate 60 that defines the second flow port 62 may define a flow path through the second plate 60 for a flow progressing through the MEMS flow module 40.

The spacing between the regulator 66 and the sidewall of the second plate 60 that defines the second flow port 62 may at least contribute to the pressure regulation function of the MEMS flow module 40 in at least some respect (e.g., in accordance with discussion of the MEMS flow module 40c of FIGS. 3A-B), although such need not be the case. It should be appreciated that the resistance to flow through the space between the perimeter of the regulator 66 and the sidewall of the second plate 60 that defines the second port 62 will change with a change in the position of the regulator 66 relative to the second plate 60. As the regulator 66 becomes offset from the second plate 60, the length of the space between the regulator 66 and the second plate 60 through which a flow must pass is reduced. This reduces the overall flow resistance, and thereby accommodates a greater flow through the MEMS flow module 40. However, another spacing for purposes of providing a pressure regulation function is defined by the position of the regulator 66 relative to the first plate 50. This gap is identified by reference numeral 58 in FIG. 2D, and will be discussed in more detail below.

The regulator 66 may be of any appropriate size, shape, and/or configuration. In the illustrated embodiment, the outside perimeter of the regulator 66 and the inside perimeter of the second flow port 62 are like-shaped (e.g., substantially conformal). However, again preferably an annular spacing exists between the regulator 66 and the second plate 60 to permit flow across the second plate 60 even when the regulator 66 is disposed within the second flow port 62. Accordingly, the shape and/or size of the regulator 66 need not be substantially the same as the shape and/or size of the second flow port 62. What is important is that the regulator 66 is sized to fluidly communicate with the first flow port 52. In the illustrated embodiment, the regulator 66 (as well as the second flow port 62 of the second plate 60) is axially aligned with the first flow port 52 through the first plate 50, and is of a larger diameter than the first flow port 52. Movement of the regulator 66 relative to the first plate 50 regulates flow through the first flow port 52 in a manner that will be more fully discussed herein.

In the illustrated embodiment, the first plate 50 exists in at least one fabrication level, the second plate 60 and regulator 66 exist in at least one different fabrication level, and the third plate 80 exists in at least one further different fabrication level (e.g., the first plate 50, second plate 60 and regulator 66, and third plate 80 may be fabricated in three adjacent structural layers of the MEMS device). Specifically, the first plate 50 may be fabricated in the P₄ layer 30, the second plate 60 and regulator 66 may be fabricated in the P₃ layer 26, and the third plate 80 may be fabricated in at least the P₂ layer 22 (see FIG. 1). The MEMS flow module 40 may further include a ring 48 that is fixedly interconnected to the outside perimeter of the top surface of the first plate 50 or that surface which is opposite the second plate 60. That is, an annular portion of the first plate 50 may be “sandwiched” between the ring 48 and the second plate 60. This ring 48 may be a metallic ring that is attached to or formed on the first plate 50 after the MEMS flow module 40 has been fabricated, or, may be made from another fabrication level. Generally, the ring 48 may provide a desired interface with a housing or other structure that incorporates the MEMS flow module 40.

As will be appreciated, the various components of the MEMS flow module 40 may be formed within different layers of a MEMS structure compared to what has been described herein. Furthermore, it will be appreciated that, unless otherwise stated, the various components of the MEMS flow module 40 may be formed in a MEMS structure in a reverse order as well. However and as noted, in the embodiment shown, the second plate 60 and regulator 66 are each formed in the P₃ layer 26 and the first plate 50 is formed in the P₄ layer 30. Accordingly, upon the removal of the S₄ layer 28 by the release in this case, a spacing of approximately 2 microns may exist between the lower surface of the first plate 50 and the upper surface of the regulator 66 and second plate 60. Changing the magnitude of this spacing by an axial movement of the regulator 66 relative to the first plate 50, in response to at least a certain change in a differential pressure across the regulator 66, will accommodate a change in the flow rate through the MEMS flow module 40 accordingly.

FIG. 2B shows a cross-sectional, exploded, perspective view of the MEMS flow module 40. Specifically, FIG. 2B is a cross-section of the MEMS flow module 40 that is taken along a plane that is parallel to the first plate 50, at a location that is between the first plate 50 and the second plate 60 so as to extend through a space between the second plate 60 and a flow-restricting ring 54 (discussed in more detail below, but a structure that extends from the first plate 50 toward, but not to, the second plate 60), with the first plate 50 having been rotated or pivoted away from the second plate 60, and where the regulator 66 remains parallel with the second plate 60 within the second flow port 62. As shown, various structures may be formed during the microfabrication process to interconnect the first plate 50 and the second plate 60. More specifically, a plurality of interconnects or anchors 70 may be formed between the top surface of the second plate 60 and the bottom surface of the first plate 50. Any number of anchors 70 may be utilized, the anchors 70 may be of any appropriate size, shape, and configuration, and the anchors 70 may be disposed in any appropriate arrangement. Likewise, one or more outer annular connectors 72 (three illustrated) and one or more inner annular connectors 74 (two illustrated) are formed between the top of the second plate 60 and the bottom of the first plate 50 at a location so as to encompass the first flow port 52. As used herein, the term “annular” only means that the connectors 72, 74 extend a full 360 degrees about a common reference point, and thereby does not limit the connectors 72, 74 to having a circular configuration.

Consider the case where the second plate 60 is fabricated in the P₃ layer 26. In this case, the anchors 70 and annular connectors 72, 74 could be fabricated after the second plate 60 and regulator 66 have been patterned from the P₃ layer 26. In this regard, an annular trench may be patterned through the P₃ layer 26 that may define both the second flow port 62 and the regulator 66. In such an embodiment, the second plate 60 and regulator 66 may be free of interconnections in their common structural layer (e.g., P₃ layer 26). Once the second plate 60 and regulator 66 have been fabricated, the S₄ layer 28 may be deposited on top of both of the second plate 60 and regulator 66, as well as into the space between the second plate 60 and the regulator 66. The S₄ layer 28 may then be patterned to define a plurality of holes therein that extend down to the P₃ layer 26 to correspond with the desired cross-sectional configuration and location of the anchors 70, and the S₄ layer 28 may also be patterned to define a plurality of annular trenches that extend down to the P₃ layer 26 to correspond with desired cross-sectional configuration and location of the annular connectors 72, 74. These holes and trenches extend all the way through the S₄ layer 28 and down to the P₃ layer 26. The P₄ layer 30 may then be deposited onto the upper surface of the S₄ layer 28 and into the holes and trenches in the S₄ layer 28. This P₄ layer 30 may then be patterned to define the perimeter of the first plate 50 and the first flow port 52 extending therethrough. The anchors 70, annular connectors 72, 74, and first plate 50 are thereby fabricated from the P₄ layer 30 and exist at a common fabrication level. Accordingly, the anchors 70 and annular connectors 72, 74 fixedly interconnect the second plate 60 to the bottom surface of the first plate 50, and maintain the first plate 50 and second plate 60 in spaced relation.

The inner annular connectors 74 increase the rigidity of the MEMS flow module 40, particularly the relative position between the first plate 50 and second plate 60 at a location in proximity to the regulator 66, which may be desirable for pressure regulation purposes. The anchors 70 and the outer annular connectors 72 also increase the rigidity of the MEMS flow module 40. In addition to providing this function, the outer annular connectors 72 provide multiple, radially spaced, redundant “radial” seals for the perimeter of the MEMS flow module 40 (e.g., the outer annular connectors 72 reduce the potential for a flow exiting the MEMS flow module 40 out from between the first plate 50 and second plate 60).

The anchors 70, outer annular connectors 72, and inner annular connector 74 increase the structural rigidity of the MEMS flow module 40. Other ways of increasing the structural rigidity of the MEMS flow module 40 could be utilized as well. For instance, the first plate 50 could be disposed or fabricated directly on the second plate 60. Consider the case where the second plate 60 and regulator 66 are fabricated in the P₃ layer 26. The S₄ layer 28 could thereafter be deposited at least on the second plate 60 and regulator 66, as well as into the space between the second plate 60 and the regulator 66. The portion of the S₄ layer 28 that is on the top surface of the second plate 60 could then be removed, while the portion of the S₄ layer 28 that is on top of the regulator 66 could be retained. A subsequent deposition of the P₄ layer 30 to define the first plate 50 would thereby directly contact the second plate 60. The P₄ layer 30 could then be patterned to define a perimeter of the first plate 50 and to define the first flow port 52. Any appropriate way of increasing the rigidity of the MEMS flow module 40 could be utilized as desired/required for a given application.

FIG. 2C shows another cross-sectional, exploded, perspective view of the MEMS flow module 40. Specifically, FIG. 2C is a cross-section of the MEMS flow module 40 that is taken along a plane that is parallel to the second plate 60, at a location that is between the second plate 60 and the third plate 80, and with the second plate 60 having been rotated or pivoted away from the third plate 80. The third plate 80 includes a plurality of third flow ports 88 that extend through the third plate 80. In the illustrated embodiment, at least part of each third flow port 88 is aligned with a corresponding portion of the gap between the second plate 60 and the regulator 66, although such may not be required in all instances. Any number of third flow ports 88 may be utilized. Moreover, the third flow ports 88 may be of any appropriate size, shape, and/or configuration, and may be disposed in any appropriate arrangement on the third plate 80.

FIG. 2C also illustrates that one or more outer annular connectors 84 are formed between the top of the third plate 80 and the bottom of the second plate 60 at a location so as to encompass the third flow ports 88 and the second flow port 62 in a lateral or radial dimension. Again, the term “annular” only means that the connectors 84 extend a full 360 degrees about a common reference point, and thereby does not limit the connectors 84 to having a circular configuration. Any number of outer annular connectors 84 may be utilized. Providing multiple, radially spaced outer annular connectors 84 provides redundant radial seals in the manner of the outer annular connectors 72 that extend between and structurally interconnect the first plate 50 and the second plate 60. It should be appreciated that part of the second plate 60 could be deposited directly on or disposed in interfacing relation with the part of the third plate 80 having the annular connectors 84, at least generally in the above-discussed manner. What is important is that a portion of the third plate 80 be un-supported so that it may flex in response to at least certain changes in the differential pressure across the MEMS flow module 40, all in order to accommodate a movement of the regulator 66 and thereby a corresponding change in the flow or flow rate through the MEMS flow module 40.

In the illustrated embodiment, the outer annular connectors 84 are formed near the perimeter of the second and third plates 60, 80. This fixed perimeter allows the third plate 80, including a central portion 82 of the third plate 80, to flex relative to the second plate 60 in a manner similar to a diaphragm, as will be discussed herein. Increasing the spacing between the “innermost” outer annular connector 84 and the central portion 82 of the third plate 80 will increase the flexibility of the third plate 80, assuming no changes are made in relation to the thickness of the third plate 80. Flexing of the third plate 80 relative to the second plate 60 is transmitted to the regulator 66. Any appropriate way of transmitting the flexing of the third plate 80 to the regulator 66 may be utilized by the MEMS flow module 40. In the illustrated embodiment, a central anchor, post, or mechanical link 86 (i.e. disposed at the geometric center of the third plate 80) fixedly interconnects the central portion 82 of the third plate 80 to the regulator 66. The central anchor 86 may be of any appropriate size, shape, and/or configuration. More than one structural interconnection could be provided between the regulator 66 and the third plate 80 as well. The outer annular connectors 84 and the central anchor 86 may be formed in a manner similar to the anchors 70 and the annular connectors 72, 74 discussed above in relation to FIG. 2B (e.g., the second plate 60, the regulators 66, the outer annular connectors 84, and the central anchor 86 may be fabricated in a common level, such as in the P₃ layer 26).

When at least a certain differential pressure exists across the MEMS flow module 40, and more specifically across the regulator 66, the regulator 66 moves at least generally axially relative to the first plate 50, through a flexing of the third plate 80 relative to the outer annular connectors 84, to increase the spacing of the regulator 66 from the first plate 50. Increasing the spacing between the regulator 66 and the first plate 50 accommodates an increased flow or flow rate through the MEMS flow module 40. The MEMS flow module 40 thereby allows a flow through the first flow port 52, into the now increased spacing between the first plate 50 and the regulator 66 that accommodates the noted increased flow rate, through that portion of the second flow port 62 that is not occupied by the regulator 66, and through the plurality of third flow ports 88 of the third plate 80. Although the regulator 66 could move axially an amount so as to be completely disposed out of the second flow port 62 in the second plate 60, this is not by any means required for the MEMS flow module 40 to provide its pressure regulation function.

FIG. 2D illustrates at least certain operational principles of the regulator 66 in relation to the first plate 50 and first flow port 52. The central anchor 86, that interconnects the regulator 66 with the third plate 80, is not illustrated in FIG. 2D. As shown in FIG. 2D, the first plate 50 and regulator 66 are shown in a static or “home” position, where a pressure differential across the MEMS flow module 40 is not yet sufficient to appreciably move the regulator 66 axially away from the first plate 50 (or further toward the first plate 50 for that matter). Stated another way, a first pressure P_{H} above the first plate 50 is not sufficiently greater than a second pressure P_{L} below the regulator 66 to move the regulator 66 axially away from the first plate 50 by a deflection of the third plate 80. Stated yet another way, the orientation illustrated in FIG. 2D may exist when there is no differential pressure at all across the regulator 66. In this static or home position for the regulator 66, the second flow plate 60 and the regulator 66 are disposed in a substantially common plane in the illustrated embodiment, although such would not need to be the case. For instance, it may be possible to fabricate the second flow plate 60 and the regulator 66 in a common fabrication level, but yet have the spacing between the regulator 66 and the first plate 50 be smaller than the spacing between the second plate 60 and the first plate 50 (e.g., by having the third plate 80 bulge or flex in the direction of the first plate 50 in its static or home position (not shown)).

The first plate 50 and regulator 66 may be spaced approximately 2 microns apart in accordance with a typical spacing between adjacent structural/fabrication MEMS layers. Although this spacing may be appropriate for one or more applications of the MEMS flow module 40, one or more other applications may benefit from having a reduced flow rate through the MEMS flow module 40 with the regulator 66 being in its home position (e.g., FIG. 2D). Stated another way, having about a 2 micron spacing between the first plate 50 and the regulator 66 may not provide a sufficient resistance to a flow for one or more applications. This may be addressed by including any appropriate flow-restricting structure to provide a desired resistance to a flow with the regulator 66 being in its home position (and thereby prior to reaching a “set-point” differential pressure, where the regulator 66 will move axially away from the first plate 50 to accommodate an increased flow or flow rate through the MEMS flow module 40).

Although the MEMS flow module 40 could be configured to have any desired “set-point” in relation to the magnitude of the differential pressure that will cause the third plate 80 to start to flex to start increasing the spacing between the first plate 50 and the regulator 66, in one embodiment this set-point is zero such that at least some flexing of the third plate 80 will occur in response to any differential pressure greater than zero or when there is any change in the differential pressure for that matter.

In the illustrated embodiment and as illustrated in FIGS. 2B and 2D, an annular flow-restricting ring 54 cooperates with the regulator 66 to provide the desired degree of flow resistance with the regulator 66 being in the home position of FIG. 2D. “Annular” again means that the flow-restricting ring 54 extends a full 360 degrees about a common point, and does not limit the flow-resisting ring 54 to having a circular configuration. Other types of flow-restricting structures could be utilized as well. For instance, the flow-restricting ring 54 could be replaced by a plurality of flow-restricting segments of any appropriate size/shape/configuration, where adjacent pairs of flow-restricting segments would be appropriately spaced from each other. The gap between such flow-restricting segments and the regulator 66, as well as the gap between each adjacent pair of flow-restricting segments, would provide the desired degree of flow restriction with the regulator 66 being in the home position of FIG. 2D. Yet another option would be to form a plug or the like on the regulator 66 that is disposed adjacent to the corresponding end of the first flow port 52, or that actually extends into the first flow port 52 such that there is preferably at least a small annular space between this plug and the sidewall of the first plate 50 that defines the first flow port 52. This particular variation is disclosed in commonly owned U.S. patent application Ser. No. 11/048,195, that was filed on Feb. 1, 2005, that is entitled “MEMS FLOW MODULE WITH PIVOTING-TYPE BAFFLE,” and the entire disclosure of which is incorporated by reference herein.

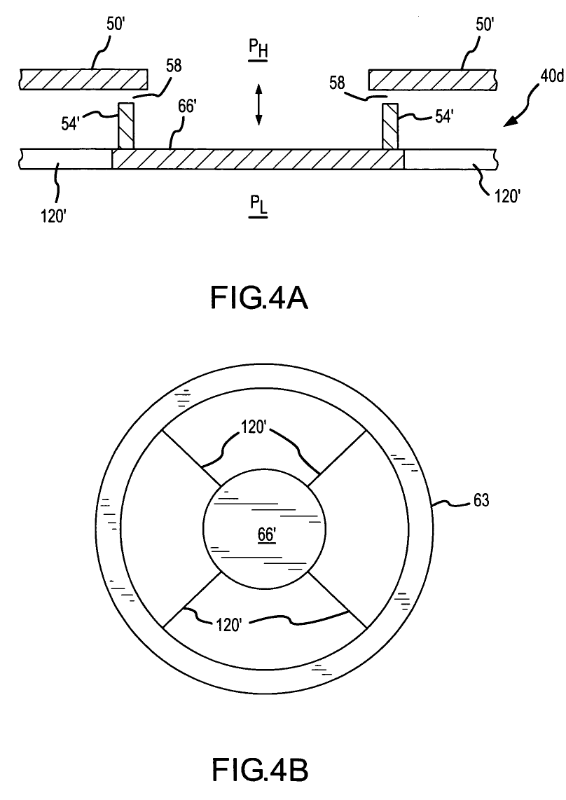

In the case where the first plate 50 is fabricated in a level that is further from the substrate 10 than the second plate 60, the annular flow-restricting ring 54 may be disposed on the bottom surface of the first plate 50 as shown, or that surface which faces the second plate 60. In the case where the first plate 50 is fabricated in a level that is closer to the substrate 10 than the second plate 60, the annular flow-restricting ring 54 may be disposed on the upper surface of the regulator 66, or that surface of the regulator 66 that faces the first plate 50 (e.g., in accordance with the MEMS flow module 40d of FIGS. 4A-B). In either case, the function of the annular flow-restricting ring 54 is to reduce the size of a flow channel between the regulator 66 and the first flow port 52. In one embodiment and with the regulator 66 being in the static or home position of FIG. 2D, the gap between the bottom of the annular flow-restricting ring 54 and the regulator 66 in the illustrated embodiment may be on the order of about 0.2 or 0.3 microns or less. Other spacing values may be appropriate, depending for instance upon the application in which the MEMS flow module 40 is being used. These same spacing values may be realized/utilized when the annular flow-restricting ring 54 instead extends from the regulator 66 in the above-noted manner. Moreover, these same spacing values may be realized/utilized when the annular flow-restricting ring 54 is replaced by a plurality of flow-restricting segments that are appropriately spaced from each other, and these same spacing values may be utilized for the spacing between each adjacent pair of flow-restricting segments.

The annular flow-restricting ring 54 may be formed in conjunction with the anchors 70 and annular connectors 72, 74. Specifically, an annular trench or trough may be formed through the S₄ layer 28 to the P₃ layer 26 on top of the regulator 66. In order to separate the annular flow-restricting ring 54 from the regulator 66, a very thin layer (e.g., about 0.2 to 0.3 microns, or even less than about 0.1 micron, but in any case corresponding with desired size of the gap 58) of sacrificial material may be deposited on top of the S₄ layer 28 and at the base of this annular trench. As will be appreciated, formation of the annular trench corresponding to the annular flow-restricting ring 54 and deposition of the thin layer of sacrificial material may be performed prior to formation of the holes and annular trenches corresponding to the anchors 70 and annular connectors 72, 74. The deposition of the thin layer of sacrificial material results, after the release, in a gap 58 between the top of the regulator 66 and the bottom or distal end of the annular flow-restricting ring 54. The thickness of the deposition may be controlled such that the resulting gap 58 (between the bottom surface of the annular flow-restricting ring 54 and the top surface of the regulator 66) substantially restricts flow through the MEMS flow module 40 in the absence of the regulator 66 being axially moved from the static or home position and away from the first plate 50. The gap 58 may also define a filter trap gap of sorts for a flow attempting to proceed between the regulator 66 and the first plate 50. In one embodiment, the gap 58 may filter a flow through the MEMS flow module 40 when the regulator 66 is in the position illustrated in FIG. 2D, while also providing a desired flow restriction through the MEMS flow module 40. Axial movement of the regulator 66 away from the first plate 50 in response to the development of at least a certain differential pressure provides a pressure regulation function in that the MEMS flow module 40 then accommodates a greater flow. When providing this pressure regulation function, the flow-restricting ring 54 may not be providing any appreciable filtering function. For at least certain applications, the primary function of the flow-restricting ring 54 is at all times to control the flow rate through the MEMS flow module 40 for purposes of providing a pressure regulation function, and not to provide any appreciable filtering function. Again, however, the flow-restricting ring 54 may provide a filtering function as desired/required.

The gap 58 may be designed such that the annular flow-restricting ring 54 and the regulator 66 are spaced to allow at least a certain flow through the MEMS flow module 40 without requiring axial movement of the regulator 66 away from the first plate 50. That is, the MEMS flow module 40 may be designed to provide a constantly open flow path that allows at least a certain limited flow through the MEMS flow module 40 at all times. Such a constantly open flow path may be beneficial in at least number of respects. One relates to the case where the MEMS flow module 40 is used to relieve intraocular pressure in an eye (e.g., by being incorporated into an eye implant). In this case, the first plate 50 of the MEMS flow module 40 could be on the “anterior chamber” side (e.g., the flow of aqueous humor out of the anterior chamber of the patient's eye through the MEMS flow module 40 would be through the first flow port 52, and then through the spacing between the regulator 66 and the first plate 50, and then ultimately out of the MEMS flow module 40 through one or more of the third flow ports 88). Having a flow path through the MEMS flow module 40 exist at all times (such that it always has a volume greater than zero, but with the flow restriction discussed herein) is believed to at least generally mimic the flow of aqueous humor out of the anterior chamber of a patient's eye through the eye's canal of Schlemm. However, the MEMS flow module 40 could be designed so that the regulator 66 is actually disposed directly on the annular flow-restricting ring 54 until at least a certain differential pressure develops (e.g., a differential pressure “set-point”), after which the regulator 66 would then move axially into spaced relation with the annular flow-restricting ring 54 to open the flow path through the MEMS flow module 40. Stated another way, the MEMS flow module 40 could be designed such that the regulator 66 is positioned to at least substantially preclude any flow through the MEMS flow module 40 until at least a certain differential pressure exists across the regulator 66.

As noted, the regulator 66 is interconnected with the “flexible” third plate 80 by the central anchor 86 in the illustrated embodiment (e.g., FIG. 2C). Generally, flexure of the third plate 80 in response to a pressure differential across the MEMS flow module 40 results in a substantially orthogonal movement of the regulator 66 relative to the plane defined by the second plate 60. More specifically, the regulator 66 moves at least generally axially or along an at least generally axial path in response to the development of at least a certain a pressure differential across the MEMS flow module 40, and more specifically across the regulator 66. If the pressure acting on the side of the regulator 66 that faces its corresponding first flow port 52 is greater than the pressure acting on the opposite side of the regulator 66 by at least a certain amount (again, including any differential pressure greater than zero), this pressure differential will result in a force that is applied to the regulator 66 that is operative to push the regulator 66 downward in the view shown in FIG. 2D by a flexing of the third plate 80. That is, the third plate 80 flexes or bulges at least generally away from the second plate 60 to allow the regulator 66 to move axially away from the first plate 50 and the annular flow-restricting ring 54 to further open/define a flow path segment within the MEMS flow module 40. This flexing also stores forces or creates stresses in the third plate 80 that may be used to return the regulator 66 either back toward or to the static/home position illustrated in FIG. 2D as the magnitude of the noted pressure differential is subsequently reduced. That is, the third plate 80 preferably elastically deforms as the pressure differential increases above a certain amount, and the elasticity of the third plate 80 may provide a restoring force that at least contributes to the axial movement of the regulator 66 back toward or to its static or home position (e.g., FIG. 2D), depending upon the magnitude of the reduction of the noted pressure differential.

The volume of a flow path segment within the MEMS flow module 40 is at least partially dependent upon the axial position of the regulator 66. The further the regulator 66 is axially displaced away from the first flow port 52, the greater the volume of the flow path segment will be (e.g., possibly up to a certain maximum). The maximum distance that the regulator 66 is allowed to move axially away from the first plate 50 may be controlled or limited, such as by using an appropriate travel limiter or the like (e.g., a mechanical “catch” that would limit how far the regulator 66 could move away from the first plate 50). Importantly, the axial movement of the regulator 66 allows the flow rate through the first flow port 52 to increase greater than proportionally to an increase in a pressure differential across the MEMS flow module 40. Stated another way, the development of at least a certain change in the differential pressure across the regulator 66 will preferably provide an increase in the volume of a flow path segment within the MEMS flow module 40 that is defined in part by the position of the regulator 66, thereby providing greater than a linear increase in the flow or flow rate through the MEMS flow module 40.

Typically the MEMS flow module 40 will be used in an application where a high pressure source P_{H} (e.g., the anterior chamber of a patient's eye—FIG. 2D) acts on the top of the regulator 66 or that surface of the regulator 66 which projects or faces toward the first plate 50, while a typically lower pressure source P_{L} (e.g., a “drainage” region outside of the eye, or within the eye or body) acts on the bottom of the regulator 66 or that surface of the regulator 66 which projects away from the first plate 50. A change in the pressure from the high pressure source P_{H} may cause the regulator 66 to axially move further away from the first plate 50, which thereby increases the flow rate through the MEMS flow module 40. Preferably, a very small change in the pressure from the high pressure source P_{H} will allow for greater than a linear change in the flow rate out of the MEMS flow module 40 through the first flow port 52, past the regulator 66 and through the second flow port 62 in the second plate 60, and in the illustrated embodiment through one or more of the plurality of third flow ports 88 through the third plate 80. For instance, a small increase in the pressure of the high pressure source P_{H} may axially move the regulator 66 (i.e., such that the regulator 66 axially moves further away from the annular flow-restricting ring 54) to provide more than a linear increase in the flow rate through the MEMS flow module 40. That is, there is preferably a non-linear relationship between the flow rate passing through the MEMS flow module 40 and a change in the differential pressure across the MEMS flow module 40 (again, more specifically the differential pressure being experienced by the regulator 66). The flow rate through the flow path segment defined between the regulator 66 and the annular flow-restricting ring 54 should be a function of the cube of the height of this flow path segment, or the extent of the gap 58 between the regulator 66 and the annular flow-restricting ring 54 (at least in the case of laminar flow, which is typically encountered at these dimensions and flow rates). Stated another way, the development of at least a certain change in the differential pressure across the regulator 66 will provide an increase in the volume of the flow channel segment between the flow-restricting ring 54 and the regulator 66, thereby providing more than a linear increase in the flow or flow rate through the MEMS flow module 40.

Consider the case where the MEMS flow module 40 is used in an implant to regulate the pressure in the anterior chamber of a patient's eye that is diseased, and where it is desired to maintain the pressure within the anterior chamber of this eye at about 5 mm of Hg. The stiffness of the third plate 80 may be configured such that it will adjust the flow rate out of the anterior chamber and through the MEMS flow module 40 such that the maximum pressure within the anterior chamber of the patient's eye should be no more than about 7-8 mm of Hg (throughout the range for which the MEMS flow module 40 is designed). Stated another way, the stiffness of the third plate 80 may allow for maintaining at least a substantially constant pressure in the anterior chamber of the patient's eye (the high pressure source P_{H} in this instance), at least for a reasonably anticipated range of pressures within the anterior chamber of the patient's eye.

In order to regulate the pressure differential across and/or flow through the MEMS flow module 40, one or more characteristics of the flow port 52 and/or third plate 80 may be adjusted. As will be appreciated, the force applied to the regulator 66 is proportional to the area of the first flow port 52 through the first plate 50. Accordingly, by adjusting the size (e.g., diameter) of the first flow port 52, the force applied to the regulator 66 for a given pressure differential may be increased and/or decreased. Likewise, the stiffness of the third plate 80 may be designed for the requirements of a particular application. The stiffness of the third plate 80 of course affects when/how the regulator 66 moves in response to experiencing a differential pressure.

There are a number of features and/or relationships that contribute to the pressure regulation function of the MEMS flow module 40, and that warrant a summarization. First is that the MEMS flow module 40 is an autonomous or self-contained device. No external power is required for operation of the MEMS flow module 40. Stated another way, the MEMS flow module 40 is a passive device—no external electrical signal of any type need be used to move the regulator 66 relative to the first plate 50 for the MEMS flow module 40 to provide its pressure regulation function. Instead, the position of the regulator 66 relative to the first plate 50 is dependent upon the differential pressure being experienced by the regulator 66, and the flow rate out of the MEMS flow module 40 is in turn dependent upon the position of the regulator 66 relative to the first plate 50 (the spacing therebetween, and thereby the size of a flow path segment of the flow path through the MEMS flow module 40). Finally, it should be noted that the MEMS flow module 40 may be designed for a laminar flow therethrough, although the MEMS flow module 40 may also be applicable for a turbulent flow therethrough as well.

The flexibility of the third plate 80 of the MEMS flow module 40 contributes to the ability of the regulator 66 to move in response to at least a certain differential pressure across the regulator 66. The size of the “unsupported” portion of the third plate 80 (i.e., the distance from the innermost annular support 84 and the center of the third plate 80) has an effect on its flexibility, as well as its thickness. Other options exist for allowing the regulator 66 to compliantly move at least generally axially relative to the first plate 50. FIGS. 2E and 2F illustrate two alternate embodiments of a third plate that may be utilized by the MEMS flow module 40 in place of the above-noted third plate 80, and therefore the MEMS flow modules of FIGS. 2E and 2F are identified by reference numerals 40a and 40b, respectively. All other features/aspects discussed above in relation to the MEMS flow module 40 of FIGS. 2A-D may be used by the MEMS flow modules 40a and 40b of FIGS. 2E and 2F, respectively. Utilization of the embodiments of FIGS. 2E and 2F may allow for providing a stiffer or more compliant support for the regulator 66, such that the sensitivity of the MEMS flow module 40 to a change in differential pressure may be increased and/or decreased accordingly.

FIG. 2E illustrates a third plate 100 that includes an outer annular support 102 for the MEMS flow module 40a. This outer annular support 102 could be interconnected with the second plate 60 in the same manner as the third plate 80 discussed above in relation to the embodiment of FIGS. 2A-D (e.g., using at least one annular connector 84; by fabricating the second plate 60 directly on the outer annular support 102 of the third plate 100). In the embodiment shown in FIG. 2E, the outer annular support 102 has an inside diameter that is distally spaced in a lateral or radial dimension from the outside diameter of the second flow port 62 through the second plate 60. The third plate 100 further includes a plurality of support members 120 that extend between the inside perimeter of the annular support 102 and converge at a central support 122. The central support 122 may be interconnected with the regulator 66 in any appropriate manner. For instance, the central support 122 could be interconnected with the regulator 66 in the same manner as described above in relation to FIG. 2C (e.g., using a central anchor 86 that extends between the central support 122 and the regulator 66). Another option would be for the support members 120 to be interconnected directly to the regulator 66.

As shown, the third plate 100 includes four support members 120 that are equally spaced from each other, and each support member 120 is disposed along a radii emanating from a common point. The space between each adjacent pair of support members 120 accommodates a flow through the third plate 100, and thereby functions as a third flow port. It will be appreciated that the number and spacing of the support members 120, as well as their size, shape, and configuration, may be selected to achieve a desired compliancy for the regulator 66. In operation, the support members 120 may support the regulator 66 in substantially co-planar relationship with the second plate 60 with the differential pressure across the MEMS flow module 40a being less than a certain amount (including where there is no differential pressure). When at least a certain pressure differential exists across the MEMS flow module 40a (again, including upon the development of any differential pressure greater than zero), the support members 120 deflect/flex to permit the regulator 66 to move orthogonally relative to the plane defined by the second plate 60 (e.g., to allow the regulator 66 to move axially away from the first plate 50).

FIG. 2F illustrates a third plate 110 for the MEMS flow module 40b that may be used in place of the third plate 80 of the MEMS flow module 40 of FIGS. 2A-D to compliantly support the regulator 66. Similar to the embodiment of FIG. 2E, the third plate 110 utilizes a plurality of support members 120 that extend from what may be characterized as an annular perimeter portion 102′ of the third plate 110 to compliantly support the regulator 66 relative to the first plate 50 and second plate 60. The annular perimeter portion 102′ may be interconnected with the second plate 60 in the same manner as the third plate 80 (e.g., using one or more annular connectors 84; by fabricating the second plate 60 directly on the annular perimeter portion 102′ of the third plate 100). Any appropriate number of support members 120 may be utilized, and each support member 120 may be of any appropriate size, shape, and configuration.

The third plate 110 also includes a plurality of wedges 114. Each wedge 114 extends from the annular perimeter portion 102′ to an inner perimeter 115 of the third plate 110 at a location that is between adjacent pairs of support members 120. Each wedge 114 is spaced from its corresponding support member 120 by a channel 116 that extends completely through the third plate 110, and each channel 116 accommodates a flow through the third plate 110. The inner perimeter 115 associated with each wedge 114 may be aligned with or spaced radially outward from a projection of the second flow port 62 onto the third plate 110. In this regard, axial movement of the regulator 66 is preferably unimpeded by the presence of the wedges 114. Stated another way, the third plate 110 may include a plurality of channels 116 that define a plurality of support members 120 that may flex to allow the regulator 66 to move relative to the first plate 50, and further that provide at least one flow path through the third plate 110.

The channels 116 not only function as flow ports through the third plate 110, but permit the support members 120 to flex relative to the remainder of the third plate 110 to in turn allow the regulator 66 to move relative to the first plate 50. These channels 116 may be formed during patterning of the third plate 110. Of note, the use of the wedges 114 may allow for a substantial portion of the third plate 110 to be rigidly interconnected with the second plate 60. In this regard, each of the wedges 114 may be fixedly interconnected with the second plate 60 utilizing one or more anchors or other structural connections (not shown) in a manner substantially similar to that discussed above in relation to the interconnection of the first and second plates 50, 60 (e.g., utilizing a plurality of anchors 70 as discussed in relation to FIG. 2B). Another option would be to fabricate the second plate 60 directly on the wedges 114 of the third plate 110. Any way of structurally interconnecting the second plate 60 with the “stationary portions” of the third plate 110 could be utilized to achieve the desired degree of rigidity for the MEMS flow module 40b.

The annular perimeter portion 102′, the wedges 114, and the support members 120 of the third plate 110 may be fabricated from a common level (e.g., P₂ layer 22; a combination of the P₂ layer 22 and the P₁ layer 18). After depositing the structural material, a patterning operation could be undertaken to define the annular perimeter portion 102′, wedges 114, and support members 120 of the third plate 110. Stated another way, portions of the third plate 80 in the embodiment of FIGS. 2A-D could be removed (e.g., corresponding with the channels 116 and the space between the inner perimeter 115 and each of the support members 120 and central support 122) to define the third plate 110.

As in the case of the embodiment of FIGS. 2A-D, the stiffness of the third plate 100 (FIG. 2E) and the third plate 110 (FIG. 2F) may be established as desired/required for a particular application and in any appropriate manner. For instance, any appropriate number of support members 120 may be utilized, and each support member 120 may be of any appropriate size, shape, and configuration.

FIGS. 3A-3B illustrate another embodiment of a MEMS flow module that is identified by reference numeral 40c. Generally, the MEMS flow module 40c provides a pressure regulation function in a single fabrication level. In this regard, the MEMS flow module 40c includes a second plate 60 with a second flow port 62 in accordance with the foregoing. A regulator 66 is at least disposable within the second flow port 62 and is compliantly supported relative to the second plate 60 in any appropriate manner (e.g., in accordance with any of the MEMS flow modules 40, 40a, 40b discussed above). The MEMS flow module 40c could include one or more additional layers that are appropriately structurally interconnected with or disposed on the second plate 60 in order to increase the rigidity of the MEMS flow module 40c (not shown).

Generally, what may be characterized as a pressure-regulating flow port 61 corresponds with the gap or space between the regulator 66 and the second plate 60 through which a flow must pass in order to progress through the MEMS flow module 40c. This flow port 61 may exist between at least part of the perimeter of the regulator 66 and a corresponding portion of the sidewall of the second plate 60 that defines the second flow port 62. That is, the flow port 61 may be characterized as corresponding with the portion of the second flow port 62 that is not occupied by the regulator 66. In this case, preferably the flow port 61 is annular in that it exists between the entire perimeter of the regulator 66 and the sidewall of the second plate 60 that defines the second flow port 62. This annular flow port 61 may be of at least a substantially constant width about the entire perimeter of the regulator 66, along its entire length, or both. For instance, the sidewall of the second plate 60 that defines the second flow port 62 may be cylindrical or frustumly-shaped or conically-shaped (e.g., tapered). One or more sidewall configurations may provide one or more desired flow characteristics. For instance, the sidewall of the second plate 60 that defines the second flow port 62 may be shaped to provide a reduced flow resistance, to thereby accommodate an increased flow through the second flow port 62. The regulator 66 could also contact the sidewall of the second plate 60 that defines the second flow port 62 at one or more locations, such that there would actually be a plurality of pressure-regulating flow ports 61 that are disposed about the perimeter of the regulator 66 (not shown).

The MEMS flow module 40c provides a pressure regulation function in either direction, as indicated by the double-headed arrow in FIG. 3A. That is, the “high-pressure source” need not be positioned on any particular side of the MEMS flow module 40c. This obviously significantly reduces the chances for an “installation error” when incorporating the MEMS flow module 40c in a particular flow path. This also of course allows the MEMS flow module 40c to be used in applications where it is desired to provide a bidirectional pressure-regulation function. Generally, changing the position of the regulator 66 relative to the second plate 60 changes the amount of resistance encountered by a flow passing through the flow port 61 by changing at least one dimension of the flow port 61. This then accommodates different flows or flow rates through the MEMS flow module 40c. More specifically, the flow resistance through the flow port 61 decreases the further the regulator 66 moves relative to the second plate 60, which accommodates an increased flow or flow rate through the MEMS flow module 40c.

FIG. 3A illustrates what may be characterized as a “home” position for the regulator 66 (e.g., when there is no differential pressure across the regulator 66, although it may be possible for the regulator 66 to be compliantly supported so as to have a differential pressure set-point other than zero in accordance with the foregoing). At this time, the pressure-regulating flow port 61 is of a maximum length (l₁), and thereby there is a maximum resistance to a flow through the flow port 61. The development of at least a certain differential pressure (“certain” again being any desired value, but preferably any differential pressure greater than zero) across the regulator 66 will cause the regulator 66 to move at least generally along an axial path away from the high-pressure side or source. One example of this case is illustrated in FIG. 3B, where the regulator 66 has moved away from a high-pressure source P_{H} in the direction of a low-pressure source P_{L}, at least generally along an axial path. This relative movement between the regulator 66 and the second plate 60 reduces the length of the pressure-regulating flow port 61 (now represented by l₂ in FIG. 3B, which is less than l₁ from FIG. 3A), and thereby reduces the flow resistance through this flow port 61. This in turn accommodates an increased flow or flow rate through the MEMS flow module 40c. A subsequent reduction in the differential pressure across the regulator 66 will cause the regulator 66 to move from the position illustrated in FIG. 3B at least back toward the home position of FIG. 3A, depending of course upon the amount of the reduction.

Changing the length of the flow port 61 while the regulator 66 remains at least partially disposed within the second flow port 62 accommodates a different flow or flow rate through the MEMS flow module 40c as noted. It should be appreciated that the regulator 66 could in fact move such that it would be completely disposed out of the second flow port 62 in the second plate 60 to accommodate a further increase in the flow or flow rate through the MEMS flow module 40c. One could say that the length of the flow port 61 reaches a minimum value once the regulator 66 is completely disposed out of the second flow port 62 (including where the top surface of the regulator 66 is coplanar with the lower surface of the second plate 60, or where the bottom surface of the regulator 66 is coplanar with the upper surface of the second plate 60), and that any further movement of the regulator 66 at least generally away from the second plate 60 will now increase the width of the flow port 61 to accommodate yet a further increase in the flow or flow rate through the MEMS flow module 40c.

FIG. 3C is an enlarged view of the portion of the second plate 60 having the second flow port 62. A sidewall 64 of the second plate 60 that defines the perimeter of this second flow port 62 is a cylindrical surface. Other configurations for the sidewall 64 may be desirable for one or more purposes. For instance, it may be possible to shape the sidewall 64 to achieve one or more desired flow characteristics. Various options are illustrated in FIGS. 3D-H. Common components between the various embodiments are identified by the same reference numeral, but a “superscript” is provided to identify the existence of at least one difference. These configurations may be used in relation to any of the flow ports discussed herein, including to define the first flow port 52 of the first plate 50. However, these configurations are particularly appropriate for when a regulator is disposable therein.

FIG. 3D illustrates that the sidewall 64ⁱ of the second plate 60ⁱ is a tapered, planar surface. Generally, the second flow port 62ⁱ has a minimum diameter at its upper extreme in the view presented in FIG. 3D, and its diameter progressively increases proceeding toward its lower extreme in the view presented in FIG. 3D. If a regulator 66 is disposed within the second flow port 62ⁱ and moves axially in either direction in response to the development of a differential pressure, the corresponding MEMS flow module should allow a larger flow or flow rate compared to the FIG. 3C configuration, even though the regulator 66 in each case may move the same amount. Generally, the spacing between the perimeter of the regulator 66 and the sidewall 64ⁱ will be greater than the spacing between the perimeter of the regulator 66 and the sidewall 64, assuming that the regulator 66 in each case starts out in the same position and moves the same amount.

FIG. 3E illustrates that the sidewall 64ⁱⁱ of the second plate 60ⁱⁱ is an arcuate surface (e.g., defined by a single radius of curvature; semi-circular). Generally, the second flow port 62ⁱⁱ has a minimum diameter midway between its upper and lower extremes in the view presented in FIG. 3E, and its diameter progressively increases proceeding away from this location in either direction. If a regulator 66 is disposed within the second flow port 62ⁱⁱ and moves axially in either direction in response to the development of a differential pressure, the corresponding MEMS flow module should allow a larger flow or flow rate compared to the FIG. 3C configuration, even though the regulator 66 in each case may move the same amount. Generally, the spacing between the perimeter of the regulator 66 and the sidewall 64ⁱⁱ will be greater than the spacing between the perimeter of the regulator 66 and the sidewall 64, assuming that the regulator 66 in each case starts out in the same position and moves the same amount. The “rounding” of the sidewall 64ⁱⁱ may be beneficial in one or more other respects as well.

FIG. 3F illustrates that the sidewall 64ⁱⁱⁱ of the second plate 60ⁱⁱⁱ is “rounded off” at its upper and lower extremes (e.g., defined by a single radius of curvature), but retains a cylindrical section at an intermediate location. Generally, the second flow port 62ⁱⁱⁱ has a minimum diameter at the cylindrical section, and its diameter progressively increases proceeding away from the cylindrical section in either direction. If a regulator 66 is disposed within the second flow port 62ⁱⁱⁱ and moves axially in either direction in response to the development of a differential pressure, the corresponding MEMS flow module should allow a larger flow or flow rate compared to the FIG. 3C configuration, even though the regulator 66 in each case may move the same amount. Generally, the spacing between the perimeter of the regulator 66 and the sidewall 64ⁱⁱⁱ will be greater than the spacing between the perimeter of the regulator 66 and the sidewall 64, assuming that the regulator 66 in each case starts out in the same position and moves the same amount. The “rounding” of the upper and lower extremes of the sidewall 64ⁱⁱⁱ may be beneficial in one or more other respects as well.

FIG. 3G illustrates that the sidewall 64^{iv} of the second plate 60^{iv} is defined by a pair of intersecting, planar sections or surfaces. In the illustrated embodiment, these planar sections intersect midway through the thickness of the second plate 60^{iv}, although such need not be the case for all applications. Generally, the second flow port 62^{iv} has a minimum diameter at the intersection of the planar sections, and its diameter progressively increases proceeding away from this intersection in either direction. If a regulator 66 is disposed within the second flow port 62^{iv} and moves axially in either direction in response to the development of a differential pressure, the corresponding MEMS flow module should allow a larger flow or flow rate compared to the FIG. 3C configuration, even though the regulator 66 in each case may move the same amount. Generally, the spacing between the perimeter of the regulator 66 and the sidewall 64^{iv} will be greater than the spacing between the perimeter of the regulator 66 and the sidewall 64, assuming that the regulator 66 in each case starts out in the same position and moves the same amount.

FIG. 3H illustrates that the sidewall 64^{v} of the second plate 60^{v} is defined by three planar sections or surfaces. An intermediate section is cylindrical, while the upper and lower sections are tapered. Generally, the second flow port 62^{v} has a minimum diameter at the intermediate section, and its diameter progressively increases proceeding away from the intermediate section in either direction. If a regulator 66 is disposed within the second flow port 62^{v} and moves axially in either direction in response to the development of a differential pressure, the corresponding MEMS flow module should allow a larger flow or flow rate compared to the FIG. 3C configuration, even though the regulator 66 in each case may move the same amount. Generally, the spacing between the perimeter of the regulator 66 and the sidewall 64^{v} will be greater than the spacing between the perimeter of the regulator 66 and the sidewall 64, assuming that the regulator 66 in each case starts out in the same position and moves the same amount.

Another embodiment of a MEMS flow module is illustrated in FIGS. 4A-B, and is identified by reference numeral 40d. The MEMS flow module 40d is similar to the MEMS flow module 40 of FIGS. 2A-D, but there are a number of distinctions. One distinction is that the flow-restricting ring 54′ extends from the regulator 66′ toward, but not to, the first plate 50′. The size of the gap 58 between the distal end of the flow-restricting ring 54′ and the first plate 50′ changes, to in turn change the flow or flow rate through MEMS flow module 40d. However, the flow-restricting ring 54′ could instead extend from the first plate 50′ in the same manner as the MEMS flow module 40 of FIGS. 2A-D. Moreover, the flow-restricting ring 54′ could be replaced by any appropriate flow-restricting structure.

Another distinction relates to the way that the regulator 66′ is compliantly supported relative to the first plate 50′. Instead of interconnecting the regulator 66 with a flexible third plate 80 as in the case of the MEMS flow module 40 of FIGS. 2A-D, the regulator 66′ of FIGS. 4A-B is compliantly supported by a plurality of support members 120′ that extend between the regulator 66′ and an outer support 63. The space between adjacent pairs of support members 120′ may be characterized as defining a flow port that is associated with the outer support 63. The outer support 63 may be of any appropriate size, shape, and/or configuration, may be interconnected with one or more other layers, or may have one or more other layers disposed thereon to provide a desired degree of rigidity for the MEMS flow module 40d. Any appropriate number of support members 120′ may be utilized as well, and each support member 120′ may be of any appropriate size, shape, and/or configuration (e.g., in the form of a flexible beam, in the form of an appropriately-shaped spring). Preferably, each support member 120′ elastically deforms, deflects, or changes shape to allow the regulator 66′ to move at least generally along an axial path relative to the first plate 50′, which thereby changes the size of the gap 58 to in turn change the flow or flow rate through the MEMS flow module 40d to provide a desired pressure regulation function.

The outer support 63, the regulator 66′, and the support members 120′ of the MEMS flow module 40d each exist in a common fabrication level. In the illustrated embodiment, the outer support 63, regulator 66′, and the plurality of support members 120′ are coplanar when there is no differential pressure across the MEMS flow module 40d. The flow-restricting ring 54′ may also exist in a common fabrication level with the outer support 63, the regulator 66′, and the support members 120′. However and as noted above, the flow-restricting ring 54′ could exist in a common fabrication level with the first plate 50′ (and thereby utilize the configuration of the corresponding portion of the MEMS flow module 40).

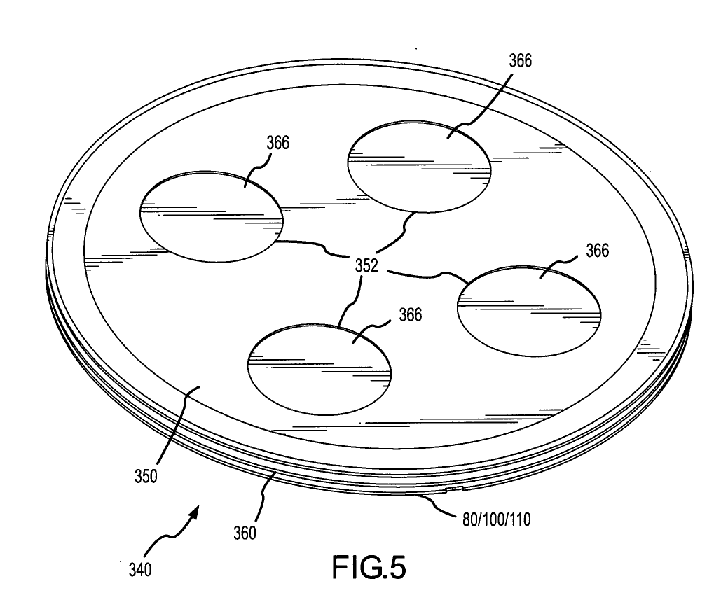

FIG. 5 shows a further embodiment of a MEMS flow module 340. The primary difference between the MEMS flow module 340 of FIG. 5 and the MEMS flow module 40 of FIGS. 2A-D is the use of multiple first flow ports 352. That is, and as shown in FIG. 5, the MEMS flow module 340 includes a first plate 350 that includes a plurality of first flow ports 352 (four) and at least a second plate 360 that includes a corresponding number of regulators 366. Any number of first flow ports 352 may be utilized, and the first flow ports 352 may be disposed in any appropriate arrangement. The MEMS flow module 340 may also utilize any third plate (e.g., 80, 100, 110) that is correspondingly adapted to compliantly support the various regulators 366. Each regulator 366 could be separately supported, two or more regulators 366 could be supported by a common structure, or each of the regulators 366 could be supported by a common structure. What is important is that the various regulators 366 are each compliantly supported such that they are able to move at least generally axially relative to the first plate 350. Axial movement of the regulators 366 relative to the first plate 350 changes the size of a flow path segment in relation to a change in a differential pressure across the MEMS flow module 340. Furthermore, the MEMS flow module 340 may include any, including all, embodiments or aspects discussed above. For instance, each flow port 352 may include an associated flow-restricting structure extending from the first plate 350 toward, but not to, the corresponding regulator 366, or vice versa.

As will be appreciated, prior to the release of the MEMS flow modules 40, 40a, 40b, 40c, 40d, and 340 discussed above, at least one sacrificial layer will be disposed between the various structures in at least certain locations. In order to remove these sacrificial layers, a plurality of etch release holes may be formed through one or more of the various structures in order to reduce the amount of time required to remove these sacrificial layers. Typically these etch release holes will have a diameter of no more than about one micron. At least certain lithographic techniques only permit the formation of an etch release hole having a diameter on the order of about one micron or more. As will be appreciated, such etch release holes will remain in the resulting MEMS flow module 40, 40a, 40b, 40c, 40d, and 340. There are a number of potential disadvantages associated with etch release holes of this size for the MEMS flow modules 40, 40a, 40b, 40c, 40d, and 340. One is that the existence of a number of etch release holes of this size may provide an undesirable a high minimum flow rate through the MEMS flow module 40, 40a, 40b, 40c, 40d, and 340 prior to reaching the differential pressure “set-point.” That is, etch release holes of this size could possibly have an undesired effect on the pressure regulating capabilities of the MEMS flow modules 40, 40a, 40b, 40c, 40d, and 340. Another is that potentially undesirable contaminants having a size about one micron or less may pass through the MEMS flow modules 40, 40a, 40b, 40c, 40d, and 340 by passing through such etch release holes.

In cases where the diameter of the etch release holes cannot be made sufficiently small (e.g., a diameter of no more than about 0.2 or 0.3 microns), and possibly depending upon the location of a particular etch release hole in the MEMS flow module 40, 40a, 40b, 40c, 40d, and 340, a flow-restricting structure or flow restrictor may be provided in relation to one or more of these etch release holes. A single flow restrictor may be associated with a single etch release hole in a given fabrication level, or may be associated with multiple etch release holes in a given fabrication level. It may be such that only a certain number of etch release holes in a given fabrication level will have an associated flow restrictor in order to provide the desired flow characteristics for the MEMS flow module 40, 40a, 40b, 40c, 40d, and 340. In any case, a flow restrictor could be used in relation to any number of etch release holes. For purposes of discussion herein, one embodiment of a flow restrictor will be described in relation to the regulator 66 of the MEMS flow module 40 of FIGS. 2A-D. However, it will be appreciated that certain aspects of the flow restrictor, including the entirety of the flow restrictor, may be applicable to other portions of the MEMS flow modules 40 and to the MEMS flow modules 40a, 40b, 40c, 40d, and 340 as well.

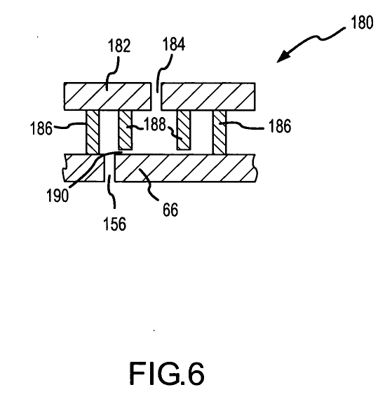

The desire to provide a restricted flow through the MEMS flow module 40, with the regulator 66 being in its “home” position (e.g., where the regulator 66 and second plate 60 are at least generally coplanar), may be especially important in biological applications, such as where the MEMS flow module 40 isolates a biological reservoir (e.g., an anterior chamber of a human eye; a cranial reservoir chamber) from another biological reservoir, the environment, and/or a man-made reservoir. In order to provide a desirable restricted flow through the MEMS flow module 40, appropriate flow restrictors may be formed for any desired etch release hole. FIG. 6 illustrates one embodiment of a flow restrictor 180 that may be formed for an etch release hole 156 through the regulator 66 and that is located on the side of the regulator 66 that faces in the direction of the first plate 50. This flow restrictor 180 is operative to provide a restricted flow through a gap 190 of about 0.1 microns or less. The size of this gap 190, and thereby the magnitude of the flow restriction, may be selected as desired/required for a particular application.

Each such flow restrictor 180 includes a top plate 182 (e.g., formed in the P₄ layer 30), an etch release hole 184 passing through the top plate 182, an annular retaining wall 186 interconnecting the top plate 182 with the regulator 66, and one or more flow-restricting walls 188 interconnected with the top plate 182 and extending downward towards, but not to the regulator 66. A singular flow-restricting wall 188 could be provided and in the form of an annular structure that extends 360 degrees about a reference axis to define a closed perimeter for the flow restrictor 180 (the illustrated embodiment). Multiple flow-restricting walls 188 that are appropriately spaced from each other could be utilized as well. The annular retaining wall 186 contains all flow between the etch release hole 184 in the top plate 182 and the etch release hole 156 in the regulator 66. Accordingly, the etch release hole 156 through the regulator 66 is disposed within the closed perimeter of the annular retaining wall 186. Likewise, the etch release hole 184 within the top plate 182 is also disposed within the closed perimeter of the annular retaining wall 186. As noted above, current lithographic techniques may not permit creation of etch release holes 156, 184 having a sufficiently small size for purposes of the MEMS flow module 40. Accordingly, the flow restrictor 180 utilizes at least one flow-restricting wall 188 that is disposed within or inwardly of the annular retaining wall 186 to provide a desired flow restriction (and to limit the size of particulates/contaminants that may pass through the flow restrictor 180 as desired/required).

As shown, each flow-restricting wall 188 is fixedly interconnected to the bottom surface of the top plate 182. As with the annular retaining wall 186, the flow-restricting wall 188 may be an annular structure that extends 360 degrees about a reference axis to define a closed perimeter. In the embodiment shown, the etch release hole 184 through the top plate 182 is disposed within or inwardly of the closed perimeter of the annular flow-restricting wall 188, while the etch release hole 156 through the regulator 66 is disposed outside or outwardly from the closed perimeter of the annular flow-restricting wall 188. The reverse of course could be done as well. The annular flow-restricting wall 188 extends downwardly towards the surface of the regulator 66, but does not contact that surface. That is, a gap 190 exists between the top of the regulator 66 and the lower edge or distal end of the annular flow-restricting wall 188. This gap 190 provides the desired flow restriction for the flow restrictor 180.

As with the annular flow-restricting ring 54 discussed above (e.g., in relation to FIGS. 2A-2D), the size of this gap 190 can be finely controlled for each flow restrictor 180 to provide a desired flow restriction (and also to provide a spacing that may reduce the potential for undesired contaminants passing completely through the flow restrictor 180 if desired/required). Accordingly, the flow restrictor 180 is formed in a manner similar to the annular flow-restricting ring 54 discussed above. In this regard and in one embodiment, once the regulator 66 is patterned, a sacrificial layer (e.g., S₄ layer 28) may be deposited on the upper surface of the regulator 66. A plurality of annular trenches or troughs may be formed in the sacrificial layer that extend all the way down to the surface of the regulator 66. These annular trenches will form the annular flow-restricting wall 188 for the various flow restrictors 180. A very thin layer of sacrificial material, for example a 0.1 micron layer, may then be deposited at the base of the annular troughs. This thin layer of sacrificial material dictates the spacing between the bottom of the annular flow-restricting wall 188 and the top surface of the regulator 66 after the release (i.e., defines the height of the gap 190). Once the thin layer of sacrificial material is deposited, a second set of annular trenches or troughs may be formed in the sacrificial layer, that again extend all the way down to the surface of the regulator 66. These additional annular trenches or troughs will form the outer retaining walls 186 for the various flow restrictors 180. Accordingly, the fabrication level that defines the top plate 182 (e.g., the P₄ layer 30) may then be deposited on top of the sacrificial layer (e.g., S₄ layer 28) such that the two sets of annular trenches or troughs defining the annular retaining walls 186 and annular flow-restricting walls 188 are filled and exist in the same fabrication level that forms the top plate 182 of each flow restrictor 180. This fabrication level may then be patterned to define the individual top plates 182 and etch release holes 184 for the flow restrictors 180.

In this arrangement, fluid has to flow through the etch release hole 184 in the top plate 182 within the closed perimeter of the annular flow-restricting wall 188, through the gap 190 between the bottom of the annular flow-restricting wall 188 and the top of the regulator 66, and then through the etch release hole 156 within the regulator 66, or vice versa. As will be appreciated, the construction of the flow restrictor 180 may be reversed such that the annular flow-restricting wall 188 is formed on the top surface of the regulator 66 and the gap 190 exists between the annular flow-restricting wall 188 and the bottom surface of the top plate 182. Likewise, it is a matter of design choice as to which etch release hole 184, 156 is disposed within the closed perimeter of the annular flow-restricting wall 188. What is important is that one of the etch release holes 156, 184 is disposed within the closed perimeter of the annular flow-restricting wall 188, and the other is disposed between the annular flow-restricting wall 188 and the annular retaining wall 186. That is, all flow through the flow restrictor 180 is preferably forced to pass through a gap 190 of a desired size. In any case, it may be such that the size of the gap 190 may be definable at smaller dimensions than the sizing of the etch release holes 156, 184 to provide a desired flow restriction.

Surface micromachining is the preferred technology for fabricating the above-described MEMS flow modules having a regulator that moves at least generally axially in response to experiencing at least a certain change in a differential pressure across the regulator. In this regard, the above-noted MEMS flow modules may be suspended above the substrate 10 after the release by one or more suspension tabs that are disposed about the perimeter of the MEMS flow module, that engage an appropriate portion of the MEMS flow module, and that are anchored to the substrate. These suspension tabs may be fractured or broken (e.g., by application of the mechanical force; electrically, such as by directing an appropriate current through the suspension tabs) to structurally disconnect the MEMS flow module from the substrate 10. One or more motion limiters may be fabricated and disposed about the perimeter of the MEMS flow module as well to limit the amount that the MEMS flow module may move in the lateral or radial dimension after the suspension tabs have been fractured and prior to retrieving the disconnected MEMS flow module. Representative suspension tabs and motion limiters are disclosed in commonly owned U.S. patent application Ser. No. 11/048,195, that was filed on Feb. 1, 2005, that is entitled “MEMS FLOW MODULE WITH PIVOTING-TYPE BAFFLE,” and the entire disclosure of which is incorporated by reference herein.

The various MEMS flow modules described herein may be fabricated in at least two different levels that are spaced from each other (hereafter a first fabrication level and a second fabrication level). Generally, a number of these MEMS flow modules include a first plate with at least one first flow port extending therethrough, and each first flow port has a regulator associated therewith that moves relative to the first plate. The first plate and first flow ports(s) may be fabricated at least in a first fabrication level, while each such regulator may be fabricated at least in the second fabrication level. Further, the second plate may also be fabricated in such a second fabrication level. It should be appreciated that the characterization of the first plate being in a “first fabrication level” and the regulator/second plate being in the “second fabrication level” by no means requires that the first fabrication level be that which is deposited “first”, and that the second fabrication level be that which is deposited “second.” Moreover, it does not require that the first fabrication level and the second fabrication level be immediately adjacent.

One or both of the regulator/second plate and the first plate each may exist in a single fabrication level or may exist in multiple fabrication levels. “Fabrication level” corresponds with what may be formed by a deposition of a structural material before having to form any overlying layer of a sacrificial material (e.g., from a single deposition of a structural layer or film). A deposition of a structural material in a single fabrication level may define an at least generally planar layer. Another option would be for the deposition of a structural material in a single fabrication level to define an at least generally planar portion, plus one or more structures that extend down toward, but not to, the underlying structural layer at the underlying fabrication level (e.g., the first plate 50 with an annular flow-restricting ring 54 extending downwardly therefrom). In either situation and prior to the release, in at least some cases there will be at least some thickness of sacrificial material disposed between at least a portion of the structures in adjacent fabrication levels (e.g., between the distal end of the flow-restricting ring 54 and the regulator 66).

Two or more structural layers or films from adjacent fabrication levels also could be disposed in direct interfacing relation (e.g., one directly on the other). Over the region that is to define a pair of plates, this would require removal of the sacrificial material that is deposited on the structural material at one fabrication level before depositing the structural material at the next fabrication level. Another option would be to maintain the separation between structural layers or films in different fabrication levels for a pair of plates, but provide an appropriate structural interconnection therebetween (e.g., a plurality of columns, posts, or the like extending between adjacent structural layers or films in different, spaced fabrication levels).

With further regard to fabricating the MEMS flow modules at least in part by surface micromachining, each component thereof (including without limitation any plate, regulator, etc.) may be fabricated in a structural layer or film at a single fabrication level (e.g., in P₁ layer 18; in P₂ layer 22; in P₃ layer 26; in P₄ layer 30 (FIG. 1 discussed above)). Consider the case of the first plate 50 of the MEMS flow module 40 of FIGS. 2A-D. The annular flow-restricting ring 54 could be fabricated by forming the second plate 60 and regulator 66 in the P₃ layer 26, depositing the S₄ layer 28, forming annular trenches or troughs in the S₄ layer 28 that extend all the way down to the P₃ layer 26, depositing sacrificial material in the bottom of these annular troughs (the thickness of which will define the spacing between the annular flow-restricting ring 54 and the regulator 66 illustrated in FIG. 2D), and then depositing the P₄ layer 30 on top of the S₄ layer 28, as well as into the “partially filled” annular troughs in the S₄ layer 28. The deposition of structural material into these “partially filled” annular troughs in the S₄ layer 28 is then what defines the annular flow-restricting ring 54. The first plate 50 and the annular flow-restricting ring 54 may then be characterized as existing in a single fabrication level (P₄ layer 30 in the noted example), since they were both defined by a deposition of a structural material before having to form any overlying layer of a sacrificial material (e.g., from a single deposition of a structural layer or film). It should be noted that at least part of the S₄ layer 28 remains between the entirety of the annular flow-restricting ring 54 and the regulator 66 (prior to the release).

Each such component of the MEMS flow modules 40, 40a, 40b, 40c, 40d, and 340 described herein could also be fabricated in multiple structural layers or films at multiple fabrication levels as noted. For instance, a plate of a given MEMS flow module could be fabricated in both the P₂ layer 22 and P₁ layer 18, where the P₂ layer 22 is deposited directly on the P₁ layer 18. Another option would be to form a particular component of a given MEMS flow module in multiple structural layers or films at different fabrication levels, but that are structurally interconnected in an appropriate manner as noted (e.g., by one or more posts, columns or the like extending between). For instance, the third plate 80 could be formed in both the P₂ layer 22 and the P₁ layer 18 with one or more structural interconnections extending therebetween (that would pass through the S₂ layer 20). Generally, this can be done by forming appropriate cuts or openings down through the S₂ layer 20 (to expose the underlying P₁ layer 18 and that will define such structural interconnections once the P₂ layer 22 is deposited therein) before depositing the P₂ layer 22.

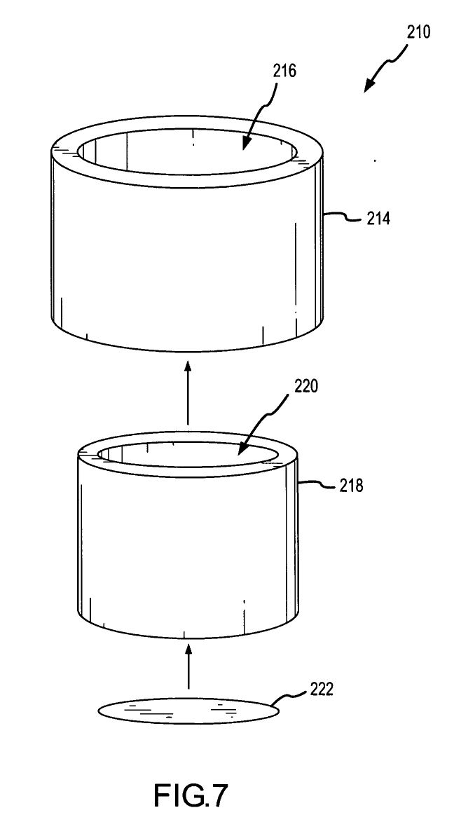

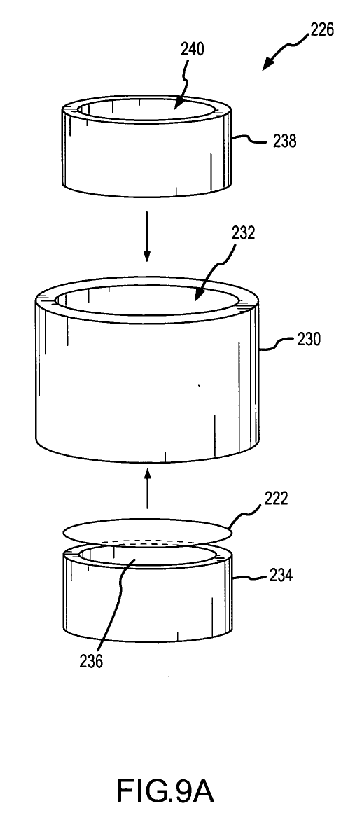

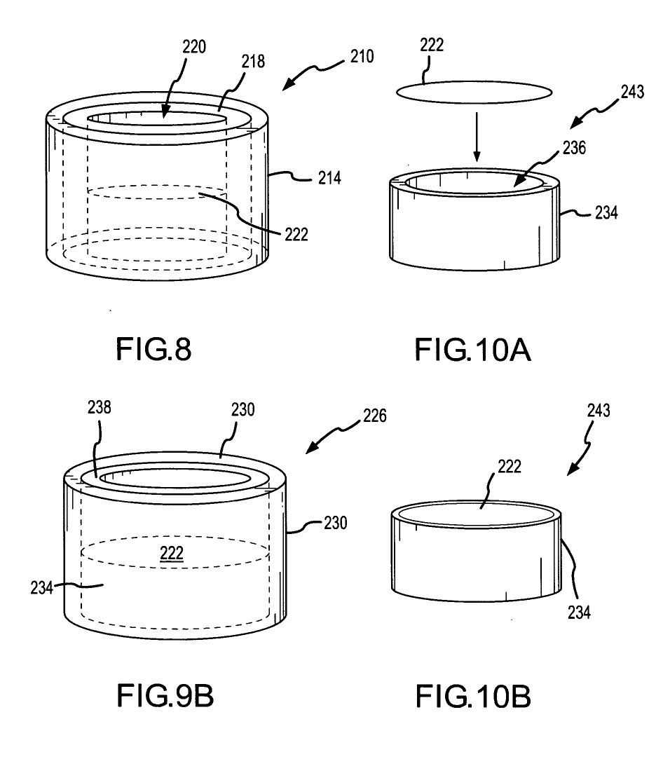

FIGS. 7-8 schematically represent one embodiment of a flow assembly 210 that may be used for any appropriate application (e.g., the flow assembly 210 may be disposed in a flow of any type, may be used to filter and/or control the flow of a fluid of any type, may be located in a conduit that fluidly interconnects multiple sources of any appropriate type (e.g., between multiple fluid or pressure sources (including where one is the environment), such as a man-made reservoir, a biological reservoir, the environment, or any other appropriate source, or any combination thereof). One example would be to dispose the flow assembly 210 in a conduit extending between the anterior chamber of an eye and a location that is exterior of the cornea of the eye. Another example would be to dispose the flow assembly 210 in a conduit extending between the anterior chamber of an eye and another location that is exterior of the sclera of the eye. Yet another example would be to dispose the flow assembly 210 in a conduit extending between the anterior chamber of an eye and another location within the eye (e.g., into Schlemm's canal) or body. In each of these examples, the conduit would provide an exit path for aqueous humor when installed for a glaucoma patient. That is, each of these examples may be viewed as a way of treating glaucoma or providing at least some degree of control of the intraocular pressure.

Components of the flow assembly 210 include an outer housing 214, an inner housing 218, and a MEMS flow module 222. Any of the MEMS flow modules described herein may be used in place of the MEMS flow module 222, including without limitation MEMS flow modules 40, 40a, 40b, 40c, 40d, and 340. The position of the MEMS flow module 222 and the inner housing 218 are at least generally depicted within the outer housing 214 in FIG. 8 to show the relative positioning of these components in the assembled condition—not to convey that the outer housing 214 needs to be in the form of a transparent structure. All details of the MEMS flow module 222 and the inner housing 218 are not necessarily illustrated in FIG. 8.

The MEMS flow module 222 is only schematically represented in FIGS. 7-8, and provides at least one of a filtering function and a pressure regulation function. The MEMS flow module 222 may be of any appropriate design, size, shape, and configuration, and further may be formed from any material or combination of materials that are appropriate for use by the relevant microfabrication technology. Any appropriate coating or combination of coatings may be applied to exposed surfaces of the MEMS flow module 222 as well. For instance, a coating may be applied to improve the biocompatibility of the MEMS flow module 222, to make the exposed surfaces of the MEMS flow module 222 more hydrophilic, to reduce the potential for the MEMS flow module 222 causing any bio-fouling, or any combination thereof. In one embodiment, a self-assembled monolayer coating (e.g., poly-ethylene-glycol) is applied in any appropriate manner (e.g., liquid or vapor phase, with vapor phase being the preferred technique) to all exposed surfaces of the MEMS flow module 222. The main requirement of the MEMS flow module 222 is that it is a MEMS device.

The primary function of the outer housing 214 and inner housing 218 is to provide structural integrity for the MEMS flow module 222 or to support the MEMS flow module 222, and further to protect the MEMS flow module 222. In this regard, the outer housing 214 and inner housing 218 each will typically be in the form of a structure that is sufficiently rigid to protect the MEMS flow module 222 from being damaged by the forces that reasonably could be expected to be exerted on the flow assembly 210 during its assembly, as well as during use of the flow assembly 210 in the application for which it was designed.

The inner housing 218 includes a hollow interior or a flow path 220 that extends through the inner housing 218 (between its opposite ends in the illustrated embodiment). The MEMS flow module 222 may be disposed within the flow path 220 through the inner housing 218 in any appropriate manner and at any appropriate location within the inner housing 218 (e.g., at any location so that the inner housing 218 is disposed about the MEMS flow module 222). Preferably, the MEMS flow module 222 is maintained in a fixed position relative to the inner housing 218. For instance, the MEMS flow module 222 may be attached or bonded to an inner sidewall or a flange formed on this inner sidewall of the inner housing 218, a press-fit could be provided between the inner housing 218 and the MEMS flow module 222, or a combination thereof. The MEMS flow module 222 also could be attached to an end of the inner housing 218 in the manner of the embodiment of FIGS. 10A-B that will be discussed in more detail below.

The inner housing 218 is at least partially disposed within the outer housing 214 (thereby encompassing having the outer housing 214 being disposed about the inner housing 218 along the entire length of the inner housing 218, or only along a portion of the length of the inner housing 218). In this regard, the outer housing 214 includes a hollow interior 216 for receiving the inner housing 218, and possibly to provide other appropriate functionality (e.g., a flow path fluidly connected with the flow path 220 through the inner housing 218). The outer and inner sidewalls of the outer housing 214 may be cylindrical or of any other appropriate shape, as may be the outer and inner sidewalls of the inner housing 218. The inner housing 218 may be retained relative to the outer housing 214 in any appropriate manner. For instance, the inner housing 218 may be attached or bonded to an inner sidewall of the outer housing 214, a press-fit could be provided between the inner housing 218 and the outer housing 214, a shrink fit could be provided between the outer housing 214 and the inner housing 218, or a combination thereof.

The inner housing 218 is likewise only schematically represented in FIGS. 7-8, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the outer housing 214 in which it is at least partially disposed. In one embodiment, the illustrated cylindrical configuration for the inner housing 218 is achieved by cutting an appropriate length from hypodermic needle stock. The inner housing 218 also may be microfabricated into the desired/required shape (e.g., using at least part of a LIGA process). However, any way of making the inner housing 218 may be utilized. It should also be appreciated that the inner housing 218 may include one or more coatings as desired/required as well (e.g., an electroplated metal; a coating to improve the biocompatibility of the inner housing 218, to make the exposed surfaces of the inner housing 218 more hydrophilic, to reduce the potential for the inner housing 218 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the inner housing 218.

The outer housing 214 likewise is only schematically represented in FIGS. 7-8, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the housing or conduit in which it is at least partially disposed or otherwise mounted. The outer housing 214 also may be microfabricated into the desired/required shape (e.g., using at least part of a LIGA process). However, any way of making the outer housing 214 may be utilized. It should also be appreciated that the outer housing 214 may include one or more coatings as desired/required as well (e.g., an electroplated metal; a coating to improve the biocompatibility of the outer housing 214, to make the exposed surfaces of the outer housing 214 more hydrophilic, to reduce the potential for the outer housing 214 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the outer housing 214.

Another embodiment of a flow assembly is illustrated in FIGS. 9A-B (only schematic representations), and is identified by reference numeral 226. The flow assembly 226 may be used for any appropriate application (e.g., the flow assembly 226 may be disposed in a flow of any type, may be used to filter and/or control the flow of a fluid of any type, may be located in a conduit that fluidly interconnects multiple sources of any appropriate type (e.g., multiple fluid or pressure sources (including where one is the environment), such as a man-made reservoir, a biological reservoir, the environment, or any other appropriate source, or any combination thereof). The above-noted applications for the flow assembly 210 are equally applicable to the flow assembly to 226. The types of coatings discussed above in relation to the flow assembly 210 may be used by the flow assembly 226 as well.

Components of the flow assembly 226 include an outer housing 230, a first inner housing 234, a second inner housing 238, and the MEMS flow module 222. The MEMS flow 222 and the inner housings 234, 238 are at least generally depicted within the outer housing 230 in FIG. 9B to show the relative positioning of these components in the assembled condition—not to convey that the outer housing 230 needs to be in the form of a transparent structure. All details of the MEMS flow module 222 and the inner housings 234, 238 are not necessarily illustrated in FIG. 9B.

The primary function of the outer housing 230, first inner housing 234, and second inner housing 238 is to provide structural integrity for the MEMS flow module 222 or to support the MEMS flow module 222, and further to protect the MEMS flow module 222. In this regard, the outer housing 230, first inner housing 234, and second inner housing 238 each will typically be in the form of a structure that is sufficiently rigid to protect the MEMS flow module 222 from being damaged by the forces that reasonably could be expected to be exerted on the flow assembly 226 during its assembly, as well as during use of the flow assembly 226 in the application for which it was designed.

The first inner housing 234 includes a hollow interior or a flow path 236 that extends through the first inner housing 234. Similarly, the second inner housing 238 includes a hollow interior or a flow path 240 that extends through the second inner housing 238. The first inner housing 234 and the second inner housing 238 are disposed in end-to-end relation, with the MEMS flow module 222 being disposed between adjacent ends of the first inner housing 234 and the second inner housing 238. As such, a flow progressing through the first flow path 236 to the second flow path 240, or vice versa, passes through the MEMS flow module 222.

Preferably, the MEMS flow module 222 is maintained in a fixed position relative to each inner housing 234, 238, and its perimeter does not protrude beyond the adjacent sidewalls of the inner housings 234, 238 in the assembled and joined condition. For instance, the MEMS flow module 222 may be bonded to at least one of, but more preferably both of, the first inner housing 234 (more specifically one end thereof) and the second inner housing 238 (more specifically one end thereof) to provide structural integrity for the MEMS flow module 222 (e.g., using cyanoacrylic esters, thermal bonding, UV-curable epoxies, or other epoxies). Another option would be to fix the position the MEMS flow module 222 in the flow assembly 226 at least primarily by fixing the position of each of the inner housings 234, 238 relative to the outer housing 230 (i.e., the MEMS flow module 222 need not necessarily be bonded to either of the housings 234, 238). In one embodiment, an elastomeric material may be disposed between the MEMS flow module 222 and the first inner housing 234 to allow the first inner housing 234 with the MEMS flow module 222 disposed thereon to be pushed into the outer housing 230 (e.g., the elastomeric material is sufficiently “tacky” to at least temporarily retain the MEMS flow module 222 in position relative to the first inner housing 234 while being installed in the outer housing 230). The second inner housing 238 also may be pushed into the outer housing 230 (before, but more likely after, the first inner housing 234 is disposed in the outer housing 230) to “sandwich” the MEMS flow module 222 between the inner housings 234, 238 at a location that is within the outer housing 230 (i.e., such that the outer housing 230 is disposed about MEMS flow module 222). The MEMS flow module 222 would typically be contacted by both the first inner housing 234 and the second inner housing 238 when disposed within the outer housing 230. Fixing the position of each of the first inner housing 234 and the second inner housing 238 relative to the outer housing 230 will thereby in effect fix the position of the MEMS flow module 222 relative to the outer housing 230. Both the first inner housing 234 and second inner housing 238 are at least partially disposed within the outer housing 230 (thereby encompassing the outer housing 230 being disposed about either or both housings 234, 238 along the entire length thereof, or only along a portion of the length of thereof), again with the MEMS flow module 222 being located between the adjacent ends of the first inner housing 234 and the second inner housing 238. In this regard, the outer housing 230 includes a hollow interior 232 for receiving at least part of the first inner housing 234, at least part of the second inner housing 238, and the MEMS flow module 222 disposed therebetween, and possibly to provide other appropriate functionality (e.g., a flow path fluidly connected with the flow paths 236, 240 through the first and second inner housings 234, 238, respectively). The outer and inner sidewalls of the outer housing 230 may be cylindrical or of any other appropriate shape, as may be the outer and inner sidewalls of the inner housings 234, 238. Both the first inner housing 234 and the second inner housing 238 may be secured to the outer housing 230 in any appropriate manner, including in the manner discussed above in relation to the inner housing 218 and the outer housing 214 of the embodiment of FIGS. 7-8.

Each inner housing 234, 238 is likewise only schematically represented in FIGS. 9A-B, and each may be of any appropriate shape/configuration, of any appropriate size, and formed from any material or combination of materials in the same manner as the inner housing 218 of the embodiment of FIGS. 7-8. Typically the outer contour of both housings 234, 238 will be adapted to match the inner contour of the outer housing 230 in which they are at least partially disposed. In one embodiment, the illustrated cylindrical configuration for the inner housings 234, 238 is achieved by cutting an appropriate length from hypodermic needle stock. The inner housings 234, 238 each also may be microfabricated into the desired/required shape (e.g., using at least part of a LIGA process). However, any way of making the inner housings 234, 238 may be utilized. It should also be appreciated that the inner housings 234, 238 may include one or more coatings as desired/required as well in accordance with the foregoing.

The outer housing 230 is likewise only schematically represented in FIGS. 9A-B, and it may be of any appropriate shape/configuration, of any appropriate size, and formed from any material or combination of materials in the same manner as the outer housing 214 of the embodiment of FIGS. 7-8. Typically the outer contour of the outer housing 230 will be adapted to match the inner contour of the housing or conduit in which it is at least partially disposed or otherwise mounted. The outer housing 230 may be microfabricated into the desired/required shape (e.g., using at least part of a LIGA process). However, any way of making the outer housing 230 may be utilized. It should also be appreciated that the outer housing 230 may include one or more coatings as desired/required in accordance with the foregoing.

Another embodiment of a flow assembly is illustrated in FIGS. 10A-B (only schematic representations), and is identified by reference numeral 243. The flow assembly 243 may be used for any appropriate application (e.g., the flow assembly 243 may be disposed in a flow of any type, may be used to filter and/or control the flow of a fluid of any type, may be located in a conduit that fluidly interconnects multiple sources of any appropriate type (e.g., between multiple fluid or pressure sources, such as a man-made reservoir, a biological reservoir, the environment, or any other appropriate source, or any combination thereof). Components of the flow assembly 243 include the above-noted housing 234 and the MEMS flow module 222 from the embodiment of FIGS. 9A-B. In the case of the flow assembly 243, the MEMS flow module 222 is attached or bonded to one end of the housing 234 (e.g., using cyanoacrylic esters, thermal bonding, UV-curable epoxies, or other epoxies). The flow assembly 243 may be disposed within an outer housing in the manner of the embodiments of FIGS. 7-9B, or could be used “as is.” The above-noted applications for the flow assembly 210 are equally applicable to the flow assembly 243. The types of coatings discussed above in relation to the flow assembly 210 may be used by the flow assembly 243 as well.

One particularly desirable application for the flow assemblies 210, 226, and 243 of FIGS. 7-10B, as discussed above, is to regulate pressure within the anterior chamber of an eye. That is, they may be disposed in an exit path through which aqueous humor travels to treat a glaucoma patient. Preferably, the flow assemblies 210, 226, 243 each provide a bacterial filtration function to reduce the potential for developing an infection within the eye. Although the various housings and MEMS flow modules used by the flow assemblies 210, 226, and 243 each may be of any appropriate color, it may be desirable for the color to be selected so as to “blend in” with the eye to at least some extent.

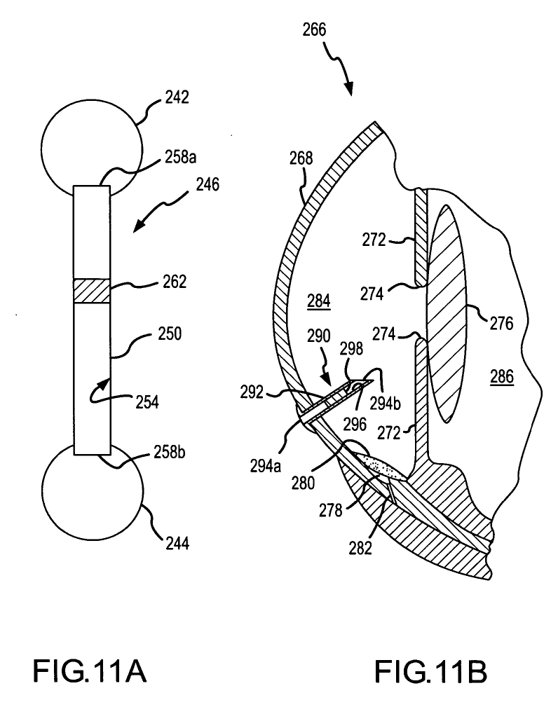

An example of the above-noted application is schematically illustrated in FIG. 11A. Here, an anterior chamber 242 of a patient's eye (or other body region for that matter—a first body region) is fluidly interconnected with an appropriate drainage area 244 by an implant 246 (a “glaucoma implant 246” for the specifically noted case). The drainage area 244 may be any appropriate location, such as externally of the eye (e.g., on an exterior surface of the cornea), within the eye (e.g., Schlemm's canal), or within the patient's body in general (a second body region).

Generally, the implant 246 includes a conduit 250 having a pair of ends 258a, 258b, with a flow path 254 extending therebetween. The size, shape, and configuration of the conduit 250 may be adapted as desired/required, including to accommodate the specific drainage area 244 being used. Representative configurations for the conduit 250 are disclosed in U.S. Patent Application Publication No. 2003/0212383, as well as U.S. Pat. Nos. 3,788,327; 5,743,868; 5,807,302; 6,626,858; 6,638,239; 6,533,768; 6,595,945; 6,666,841; and 6,736,791, the entire disclosures of which are incorporated by reference in their entirety herein.

A flow assembly 262 is disposed within the flow path 254 of the conduit 250. All flow leaving the anterior chamber 242 through the implant 246 is thereby directed through the flow assembly 262. Similarly, any flow from the drainage area 244 into the implant 246 will have to pass through the flow assembly 262. The flow assembly 262 may be retained within the conduit 250 in any appropriate manner and at any appropriate location (e.g., it could be disposed on either end 258a, 258b, or any intermediate location therebetween). The flow assembly 262 may be in the form of any of the flow assemblies 210, 226, or 243 discussed above, replacing the MEMS flow module 222 with any of the MEMS flow modules in accordance with FIGS. 1-6. Alternatively, the flow assembly 262 could simply be in the form of the MEMS flow modules in accordance with FIGS. 1-6. Any appropriate coating may be applied to at least those surfaces of the implant 246 that would be exposed to biological material/fluids, including without limitation a coating that improves biocompatibility, that makes such surfaces more hydrophilic, and/or that reduces the potential for bio-fouling. In one embodiment, a self-assembled monolayer coating (e.g., poly-ethylene-glycol) is applied in any appropriate manner (e.g., liquid or vapor phase, with vapor phase being the preferred technique) to the noted surfaces.

FIG. 11B illustrates a representative embodiment in accordance with FIG. 11A. Various portions of the eye 266 are identified in FIG. 11B, including the cornea 268, iris 272, pupil 274, lens 276, anterior chamber 284, posterior chamber 286, Schlemm's canal 278, trabecular meshwork 280, and aqueous veins 282. Here, a glaucoma implant or shunt 290 having an appropriately shaped conduit 292 is directed through the cornea 268. The conduit 292 may be in any appropriate form, but will typically include at least a pair of ends 294a, 294b, as well as a flow path 296 extending therebetween. End 294a is disposed on the exterior surface of the cornea 268, while end 294b is disposed within the anterior chamber 284 of the eye 266.

A flow assembly 298 is disposed within the flow path 296 of the conduit 292. All flow leaving the anterior chamber 284 through the shunt 290 is thereby directed through the flow assembly 298. Similarly, any flow from the environment back into the shunt 290 will have to pass through the flow assembly 298 as well. Preferably, the flow assembly 298 provides a bacterial filtration function to reduce the potential for developing an infection within the eye when using the implant 290. The flow assembly 298 may be retained within the conduit 292 in any appropriate manner and at any appropriate location (e.g., it could be disposed on either end 294a, 294b, or any an intermediate location therebetween). The flow assembly 298 may be in the form of any of the flow assemblies 210, 226, or 243 discussed above, replacing the MEMS flow module 222 with any of the MEMS flow modules in accordance with FIGS. 1-6. Alternatively, the flow assembly 298 could simply be in the form of the MEMS flow modules in accordance with FIGS. 1-6. Any appropriate coating may be applied to at least those surfaces of the shunt 290 that would be exposed to biological material/fluids, including without limitation a coating that improves biocompatibility, that makes such surfaces more hydrophilic, and/or that reduces the potential for bio-fouling. In one embodiment, a self-assembled monolayer coating (e.g., poly-ethylene-glycol) is applied in any appropriate manner (e.g., liquid or vapor phase, with vapor phase being the preferred technique) to the noted surfaces.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A MEMS flow module, comprising:
a first plate comprising a first flow port;
a second plate comprising a second flow port;
a regulator disposable within said second flow port, wherein said regulator fluidly communicates with said first flow port, wherein said second plate and said regulator are disposed in a substantially common plane in the absence of at least a certain differential pressure across said MEMS flow module, and wherein said regulator is moveable relative to each of said first and second plates to change a magnitude of a spacing of said regulator from said first plate in response to at least a certain change in a differential pressure across said MEMS flow module.

2. A MEMS flow module, as claimed in claim 1, wherein said regulator moves at least generally axially in response to at least a certain change in a differential pressure across said MEMS flow module.

3. A MEMS flow module, as claimed in claim 1, wherein said second plate and said regulator exist in a common fabrication level.

4. A MEMS flow module, as claimed in claim 3, wherein said regulator and said second plate are free of any interconnection in said common fabrication level.

5. A MEMS flow module, as claimed in claim 1, wherein said regulator has a larger diameter than said first flow port, and wherein a center of said regulator is axially aligned with a center of said first flow port.

6. A MEMS flow module, as claimed in claim 1, wherein an outside perimeter of said regulator and a sidewall of said second plate that defines said second flow port are separated by an annular gap.

7. A MEMS flow module, as claimed in claim 1, further comprising:
a first annular wall that interconnects said first plate and said second plate, wherein said first and second flow ports are located inwardly of said first annular wall in a lateral dimension.

8. A MEMS flow module, as claimed in claim 7, further comprising:
a third plate spaced from said second plate, wherein said second plate is located between said first plate and said third plate, wherein said MEMS flow module further comprises a second annular wall that interconnects said third plate and said second plate, and wherein said first and second flow ports and said regulator are also located inwardly of said second annular wall in said lateral dimension.

9. A MEMS flow module, as claimed in claim 8, wherein said third plate further comprises at least one third flow port disposed within a region defined by said second annular wall.

10. A MEMS flow module, as claimed in claim 1, further comprising:
a third plate, wherein said second plate is located between said first and third plates, wherein said third plate is interconnected with and spaced from said regulator, and wherein said third plate comprises at least one third flow port.

11. A MEMS flow module, as claimed in claim 10, wherein said third plate is in the form of a diaphragm that is flexible to allow said regulator to move away from said first plate in response to at least a certain change in a differential pressure across said MEMS flow module.

12. A MEMS flow module, as claimed in claim 10, wherein, said third plate comprises a plurality of flexible support members that are interconnected with said regulator and that flex to allow said regulator to move away from said first plate in response to at least a certain change in a differential pressure across said MEMS flow module.

13. A MEMS flow module, as claimed in claim 12, wherein said plurality of support members each extend along a radii emanating from a common point.

14. A MEMS flow module, as claimed in claim 1, wherein centers of said first flow port and said second flow port are axially aligned.

15. A MEMS flow module, as claimed in claim 1, wherein movement of said regulator away from said first plate in response to at least a certain increase in a differential pressure across said MEMS flow module accommodates an increased flow through said MEMS flow module.

16. A MEMS flow module, as claimed in claim 1, wherein said MEMS flow module further comprises at least one flow-restricting structure located within a space between said regulator and said first plate, wherein all flow through said first flow port must pass through a space defined in part by said flow-restricting structure.

17. A MEMS flow module, as claimed in claim 16, wherein said flow-restricting structure extends from said first plate toward said regulator, and terminates prior to reaching said regulator.

18. A MEMS flow module, as claimed in claim 1, wherein said first plate comprises a plurality of said first flow ports, and wherein said second plate further comprises a plurality of said second flow ports and said regulators, wherein each said first flow port has a corresponding second flow port and a corresponding said regulator.

19. A MEMS flow module, as claimed in claim 1, wherein said MEMS flow module is a passive device.

20. An implant for addressing pressure within a first body region, comprising said MEMS flow module of claim 1 and a conduit, wherein said conduit comprises a flow path that is adapted to fluidly interconnect with the first body region, and wherein said MEMS flow module is disposed in said flow path.

21. An implant, as claimed in claim 20, further comprising at least one housing, wherein said at least one housing is disposed within said conduit, and wherein said MEMS flow module interfaces with said at least one housing.

22. An implant installable in a human eye and comprising said MEMS flow module of claim 1 and a conduit, wherein said conduit comprises a flow path that is adapted to fluidly interconnect with an anterior chamber of the human eye when said implant is installed, and wherein said MEMS flow module is disposed in said flow path.

23. A MEMS flow module, comprising:
a first fabrication level comprising a first plate, wherein said first plate comprises a first flow port;
a second fabrication level comprising a second plate, a second flow port associated with said second plate, and a regulator, wherein said regulator fluidly communicates with said first flow port, and wherein said regulator is moveable relative to said first and second plates to change a magnitude of a spacing of said regulator from said first plate in response to at least a certain change in a differential pressure across said MEMS flow module.

24. A MEMS flow module, as claimed in claim 23, wherein said regulator is movable at least generally axially in response to at least a certain change in a differential pressure across said MEMS flow module.

25. A MEMS flow module, as claimed in claim 23, wherein said regulator is disposed within said second flow port in the absence of at least a certain differential pressure across said MEMS flow module.

26. A MEMS flow module, as claimed in claim 23, further comprising:
a first annular wall that interconnects said first and second plates, wherein said first and second flow ports are located inwardly of said first annular wall in a lateral dimension.

27. A MEMS flow module, as claimed in claim 23, wherein said second plate and said regulator are disposed in a substantially common plane in the absence of at least a certain differential pressure across said MEMS flow module.

28. A MEMS flow module, as claimed in claim 23, wherein an outside perimeter of said regulator and a sidewall of said second plate that defines said second flow port are separated by an annular gap when said regulator is disposed within said second flow port.

29. A MEMS flow module, as claimed in claim 23, wherein said second plate comprises an annular support, and wherein said MEMS flow module further comprises a plurality of flexible support members that movably interconnect said annular support and said regulator, wherein said plurality of flexible support members also exist in said second fabrication level.

30. A MEMS flow module, as claimed in claim 23, further comprising:
a third fabrication level, wherein said second fabrication level is located between said first and third fabrication levels, wherein said third fabrication level comprises a third plate that compliantly supports said regulator relative to use said first and second plates, and wherein said third plate comprises a third flow port.

31. A MEMS flow module, as claimed in claim 30, wherein said third plate is in the form of a diaphragm that is flexible to allow said regulator to move away from said first plate in response to at least a certain change in a differential pressure across said MEMS flow module.

32. A MEMS flow module, as claimed in claim 30, wherein said third plate comprises a plurality of flexible support members that are interconnected with said regulator and that flex to allow said regulator to move generally away from said first plate in response to at least a certain change in a differential pressure across said MEMS flow module.

33. A MEMS flow module, as claimed in claim 23, wherein said MEMS flow module further comprises at least one flow-restricting structure located within a space between said regulator and said first plate, wherein all flow through said first flow port must pass through a space defined in part by said flow-restricting structure.

34. A MEMS flow module, as claimed in claim 33, wherein said flow-restricting structure extends from said first plate toward said regulator, and terminates prior to reaching said regulator.

35. An implant for addressing pressure within a first body region, comprising said MEMS flow module of claim 23 and a conduit, wherein said conduit comprises a flow path that is adapted to fluidly interconnect with the first body region, and wherein said MEMS flow module is disposed in said flow path.

36. An implant, as claimed in claim 35, further comprising at least one housing, wherein said at least one housing is disposed within said conduit, and wherein said MEMS flow module interfaces with said at least one housing.

37. An implant installable in a human eye and comprising said MEMS flow module of claim 23 and a conduit, wherein said conduit comprises a flow path that is adapted to fluidly interconnect with an anterior chamber of the human eye when said implant is installed, and wherein said MEMS flow module is disposed in said flow path.

38. A MEMS flow module, comprising:
a first plate comprising a first flow port;
a second plate comprising a second flow port; and
a regulator to fluidly communicates with said first flow port, wherein an outside perimeter of said regulator and a sidewall of said second plate that defines said second flow port are separated by an annular gap when said regulator is disposed within said second flow port, and wherein said regulator is moveable relative to said first and second plates to change a magnitude of a spacing of said regulator from said first plate in response to at least a certain change in a differential pressure across said MEMS flow module.

39. A MEMS flow module, as claimed in claim 38, wherein said regulator moves at least generally axially in response to at least a certain change in a differential pressure across said MEMS flow module.

40. A MEMS flow module, as claimed in claim 38, further comprising:
a first annular wall that interconnects said first and second plates, wherein said first and second flow ports are located inwardly of said first annular wall in a lateral dimension.

41. A MEMS flow module, as claimed in claim 38, wherein said second plate and said regulator are disposed in a substantially common plane in the absence of at least a certain differential pressure across said MEMS flow module.

42. A MEMS flow module, as claimed in claim 38, wherein said second plate and said regulator are fabricated in a common fabrication level.

43. A MEMS flow module, as claimed in claim 42, wherein said regulator and said second plate are free of interconnections in said common fabrication level.

44. A MEMS flow module, as claimed in claim 38, further comprising:
a compliant support structure spaced from said second plate and such that said second plate is between said compliant support and said first plate, wherein said complaint support structure supports said regulator relative to said first and second plates.

45. A MEMS flow module, as claimed in claim 44, wherein said compliant support structure permits said regulator to move at least generally axially in response to at least a certain change in a differential pressure across said MEMS flow module.

46. An implant for addressing pressure within a first body region, comprising said MEMS flow module of claim 38 and a conduit, wherein said conduit comprises a flow path that is adapted to fluidly interconnect with the first body region, and wherein said MEMS flow module is disposed in said flow path.

47. An implant, as claimed in claim 46, further comprising at least one housing, wherein said at least one housing is disposed within said conduit, and wherein said MEMS flow module interfaces with said at least one housing.

48. An implant installable in a human eye and comprising said MEMS flow module of claim 38 and a conduit, wherein said conduit comprises a flow path that is adapted to fluidly interconnect with an anterior chamber of the human eye when said implant is installed, and wherein said MEMS flow module is disposed in said flow path.

49. A MEMS flow module, comprising:
a first plate comprising a first flow port;
a second plate comprising a second flow port;
a regulator that fluidly communicates with said first flow port; and
a third plate comprising a third flow port, wherein said regulator is located between said first plate and said third plate, and wherein at least a portion of said third plate compliantly supports said regulator to allow said regulator to move at least generally axially away from said first plate in response to at least a certain change in a differential pressure across said MEMS flow module to accommodate an increased flow through said MEMS flow module in a direction that proceeds through said first port, then through said second flow port, and then through said third flow port.

50. An implant installable in a human eye and comprising said MEMS flow module of claim 49 and a conduit, wherein said conduit comprises a flow path that is adapted to fluidly interconnect with an anterior chamber of the human eye when said implant is installed, and wherein said MEMS flow module is disposed in said flow path.

51. An implant, as claimed in claim 50, further comprising at least one housing, wherein said at least one housing is disposed within said conduit, and wherein said MEMS flow module interfaces with said at least one housing.

52. A MEMS flow module, comprising:
a plate comprising a flow port;
a regulator disposable within said flow port, wherein said regulator is moveable relative to said plate in response to at least a certain change in a differential pressure across said MEMS flow module.

53. A MEMS flow module, as claimed in claim 52, wherein said regulator moves at least generally axially in response to at least a certain change in a differential pressure across said MEMS flow module.

54. A MEMS flow module, as claimed in claim 52, wherein said plate and said regulator exist in a common fabrication level.

55. A MEMS flow module, as claimed in claim 54, wherein said regulator and said second plate are free of any interconnection in said common fabrication level.

56. A MEMS flow module, as claimed in claim 52, wherein an outside perimeter of said regulator and a sidewall of said plate that defines said flow port are separated by an annular gap when said regulator is disposed within said flow port.

57. A MEMS flow module, as claimed in claim 52, wherein movement of said regulator in response to experiencing at least a certain a differential pressure accommodates an increased flow through said MEMS flow module.

58. An implant for addressing pressure within a first body region, comprising said MEMS flow module of claim 52 and a conduit, wherein said conduit comprises a flow path that is adapted to fluidly interconnect with the first body region, and wherein said MEMS flow module is disposed in said flow path.

59. An implant, as claimed in claim 58, further comprising at least one housing, wherein said at least one housing is disposed within said conduit, and wherein said MEMS flow module interfaces with said at least one housing.

60. An implant installable in a human eye and comprising said MEMS flow module of claim 52 and a conduit, wherein said conduit comprises a flow path that is adapted to fluidly interconnect with an anterior chamber of the human eye when said implant is installed, and wherein said MEMS flow module is disposed in said flow path.
